(19) United States
(12) Patent Application Publication
(10) Pub. No.: US 2005/0096491 A1
(43) Pub. Date: May 5, 2005

(54) Aromatic methylidene compound, methylstyryl compound for producing the same, production method therefor, and organic electroluminescent element

(75) Inventor: Mitsuru Hashimoto, Yokohama-Shi (JP)

Correspondence Address: DARBY & DARBY P.C., P. O. BOX 5257, NEW YORK, NY, 10150-5257, US

(73) Assignee: Matsushita Electric Industrial Co., Ltd., Osaka (JP)

(21) Appl. No.: 10/996,322

(22) Filed: Nov. 23, 2004

Related U.S. Application Data
(62) Division of application No. 10/354,822, filed on Jan. 30, 2003.

(30) Foreign Application Priority Data
Feb. 6, 2002 (JP) JP 2002-028875
Feb. 6, 2002 (JP) JP 2002-028876
Feb. 6, 2002 (JP) JP 2002-028877
Feb. 6, 2002 (JP) JP 2002-028878
Feb. 6, 2002 (JP) JP 2002-028879
Feb. 6, 2002 (JP) JP 2002-028880
Feb. 6, 2002 (JP) JP 2002-028881

(51) Int. Cl. C07C 1/32; C07C 15/52; C07C 15/58; C07C 15/60; C09K 11/06; H05B 33/14

(52) U.S. Cl. 585/435; 428/690; 428/917; 313/504; 257/40; 252/301.16

(57) ABSTRACT

A compound of the formula (1): 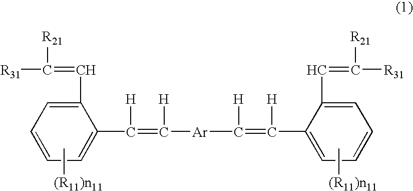
wherein R₁₁ represents an alkyl group, an alkoxy group, a halogen atom, a cyano group, or a nitro group; n₁₁ is an integer of 0 to 4; R₂₁ and R₃₁ are a hydrogen atom, an alkyl group, a cycloalkyl group, an aromatic group, or a heteroaromatic group; or R₂₁ and R₃₁ together form a condensed ring consisting of aromatic rings or heteroaromatic rings; and Ar is selected from the groups consisting of groups represented by the following formulae: 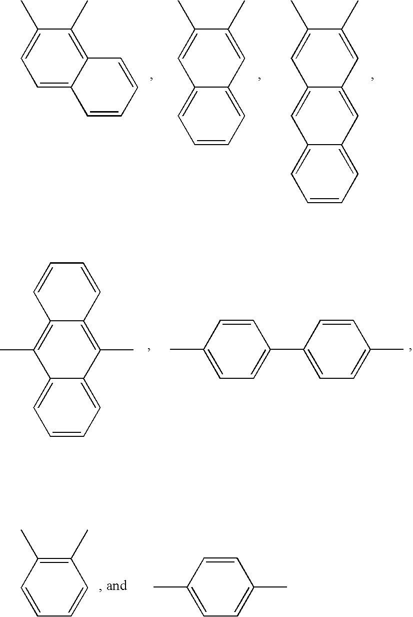 and a methylstyryl compound for producing the same, the production methods thereof and an organic electroluminescent element having the compound.

### BACKGROUND OF THE INVENTION

The present invention relates to a novel organic methylidene compound useful as functional materials for a sensor in an electronic camera, as functional materials in an organic electroluminescent element such as electric charge transporting materials and luminescent materials, and as functional materials for other various organic semiconductor elements. The present invention also relates to a methylstyryl compound useful for producing the same, production methods therefor, and an organic electroluminescent element.

An electroluminescent element utilizing an electroluminescent phenomenon of substances is self-luminescent unlike a liquid crystal element and thus has a high visibility, which results in a clear view when used as a display device. Since the electroluminescent element is completely in a solid state, the element has properties such as shock-resistant property. It is thus expected that the electroluminescent element will find a variety of uses for, e.g., a thin display, a back light of liquid crystal displays and a plane light source.

One of the electroluminescent elements that are practically used at present is a dispersion electroluminescent element containing inorganic materials such as zinc sulfide. However, the dispersion, electroluminescent element requires high a.c. voltage for driving, and therefore have problems such as complicated driving circuits and low brightness. Therefore, such an element is not widely put into practical use.

An organic electroluminescent element using organic materials has been spotlighted since C. W. Tang et al. proposed in 1987 an element having a laminate structure in -which an electron-transporting organic fluorescent substance and a positive hole-transporting organic substance are stacked and both carriers for electrons and for positive holes are injected into the fluorescent material layer to generate luminescence (C. W. Tang and S. V. VAN Slyke, Appl. Phys. Lett., Vol. 51, p. 913-915 (1987); JP-A-S63-264629) . It is reported that luminescence of at least 1000 cm⁻² was obtained under a driving voltage of not more than 10 V in the element. Following this proposal, various investigations for these materials have been carried out actively. Various materials and element structures have been proposed to date and researches for their practical use are being performed actively.

On the other hand, organic electroluminescent elements using the organic materials proposed hitherto still have various problems. For example, functions of the elements may deteriorate even during storing in either driving or non-driving state. Such deterioration may cause lowering in luminescence brightness and generation and growth of a non-luminescent region that is called dark spot in driving or non-driving state, which finally lead to a short circuit and rupture of the element. Such phenomena are considered to be essential problems in the materials used. In the present state, it is hardly recognized that the elements have sufficient lives for their practical use. Therefore, their practical use is restricted to devices in which a short life may be accepted. Further, none of the present systems and luminescent materials are suitable for an element for a color display. In order to solve these problems and to attain their wide practical use, it is an important technical object to seek for new high functional luminescent materials and electric charge transporting materials.

### SUMMARY OF THE INVENTION

The present invention has been made in view of the aforementioned present state of the art of the organic electroluminescent elements. The object of the present invention is to provide an aromatic methylidene compound that is especially useful as materials, especially an luminescent material, that may give an organic electroluminescent element having bright luminescence at low voltage and high durability. The present invention also relates to a methylstyryl compound useful for producing the same, production methods therefor, and an organic electroluminescent element having high brightness and high durability.

According to the present invention, there is provided a compound represented by the following formula (1): 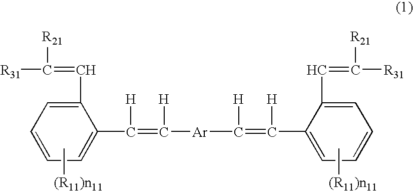
wherein R₁₁ represents a non-substituted or substituted alkyl group, a non-substituted or substituted alkoxy group, a halogen atom, a cyano group, or a nitro group, provided that, when two or more of groups R₁₁ are present, the groups R₁₁ are the same as or different from each other;
n₁l is an integer of 0 to 4;
R₂₁ and R₃₁ are the same as or different from each other and each represents a hydrogen atom provided that one of R₂₁ and R₃₁ is not hydrogen atom, a non-substituted or substituted alkyl group provided that one of R₂₁ and R₃₁ is not the alkyl group, a non-substituted or substituted cycloalkyl group provided that one of R₂₁ and R₃₁ is not the cycloalkyl group, a non-substituted or substituted aromatic group, or a non-substituted or substituted heteroaromatic group; or R₂₁ and R₃₁ together form a condensed ring consisting of non-substituted or substituted aromatic rings or non-substituted or substituted heteroaromatic rings; and
Ar is selected from the groups consisting of groups represented by the following formulae:

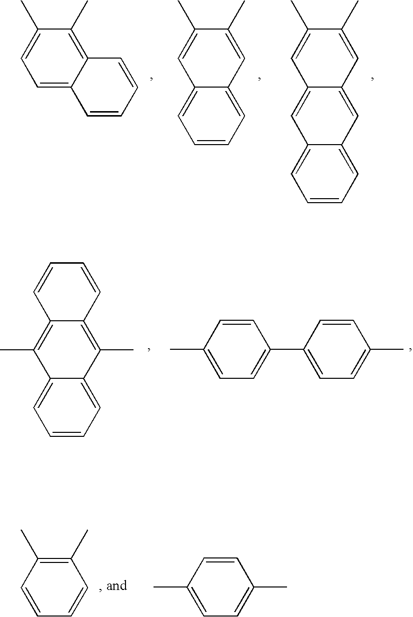

According to the present invention, there is further provided an intermediate compound for preparing the compound of the formula (1), said intermediate compound being represented by the following formula (2): 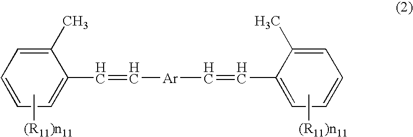
wherein R₁₁, n₁₁ and Ar are the same as those in the formula (1).

According to the present invention, there is further provided a method for producing the compound of the formula (1), said method comprising the step of reacting a compound represented by the following formula (3):
ZH₂C—Ar—CH₂Z (3)
wherein Ar is the same as that in the formula (1), and groups Z are the same as or different from each other and each represents —PO(OR)₂ or —PA₃⁺ or a salt thereof, wherein R represents a non-substituted or substituted alkyl group, and A represents a non-substituted or substituted aryl group;
with a compound represented by the following formula (4): 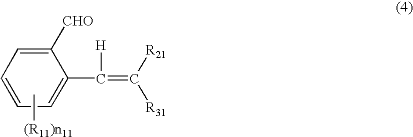
wherein R₁₁, R₂₁, R₃₁ and nil are the same as those in the formula (1).

According to the present invention, there is further provided a method for producing the compound of the formula (1), said method comprising the step of reacting a compound represented by the following formula (5): 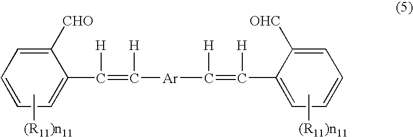
wherein R₁₁, n₃₁ and Ar are the same as those in the formula (1);
with a compound represented by the following formula (6): 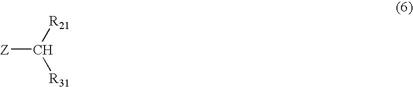
   wherein R₂₁ and R₃₁ are the same as those in the formula (1), and Z represents —PO(OR)₂ or —PA₃⁺ or a salt thereof, wherein R represents a non-substituted or substituted alkyl group, and A represents a non-substituted or substituted aryl group.

According to the present invention, there is further provided a method for producing the compound of the formula (1), said method comprising the step of reacting a compound represented by the following formula (7):
OHC—Ar—CHO (7)
wherein Ar is the same as that in the formula (1);
with a compound represented by the following formula (8): 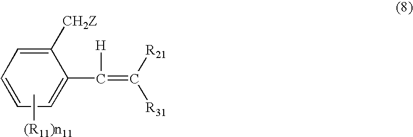
wherein R₁₁, R₂₁, R₃₁ and n₁₁ are the same as those in the formula (1), and Z represents —PO(OR)₂ or —PA₃⁺ or a salt thereof, wherein R represents a non-substituted or substituted alkyl group, and A represents a non-substituted or substituted aryl group.

According to the present invention, there is further provided a method for producing the compound of the formula (1), said method comprising the step of reacting a compound represented by the following formula (9): 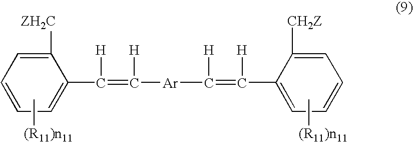
wherein R₁₁, n₁₁ and Ar are the same as those in the formula (1), and groups Z are the same as or different from each other and each represents —PO(OR)₂ or —PA₃⁺ or a salt thereof, wherein R represents a non-substituted or substituted alkyl group, and A represents a non-substituted or substituted aryl group;
with a compound represented by the following formula (10): 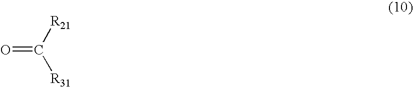
wherein R₂₁ and R₃₁ are the same as those in the formula (1) .

According to the present invention, there is further provided a method for producing the compound of the formula (2), said method comprising the step of reacting a compound represented by the following formula (3):
ZH₂C—Ar—CH₂Z (3)
wherein Ar is the same as that in the formula (1), and groups Z are the same as or different from each other and each represents —PO(OR)₂ or —PA₃⁺ or a salt thereof, wherein R represents a non-substituted or substituted alkyl group, and A represents a non-substituted or substituted aryl group;
with a compound represented by the following formula (11): 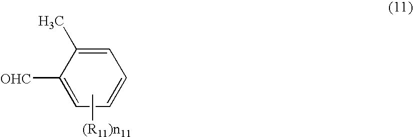
wherein R₁₁ and n₁₁ are the same as those in the formula (1).

According to the present invention, there is further provided a method for producing the compound of the formula (2), said method comprising the steps of reacting a compound represented by the following formula (7):
OHC—Ar—CHO (7)
wherein Ar is the same as that in the formula (1);
with a compound represented by the following formula (12): 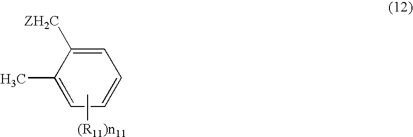
wherein R₁₁ and n₁₁ are the same as those in the formula (1); and Z represents —PO(OR)₂ or —PA₃⁺ or a salt thereof, wherein R represents a non-substituted or substituted alkyl group, and A represents a non-substituted or substituted aryl group.

According to the present invention, there is further provided an organic electroluminescent element comprising a layer containing the compound of the formula (1).

The compound represented by the formula (1) according to the present invention is useful as a constituent, particularly as a luminescent material, of an organic electroluminescent element. The compound represented by the formula (2) is an useful intermediate for producing the compound represented by the formula (1). The compounds and the production method therefor provided by the present invention make a great contribution to production of an organic electroluminescent element having high brightness and high durability.

### BRIEF DESCRIPTION OF DRAWINGS

FIG. 1 is a schematic sectional view of an element that is an embodiment of the present invention having an anode, a luminescence layer and a cathode.

FIG. 2 is a schematic sectional view of an element that is an embodiment of the present invention having an anode, a positive hole-transporting layer, a luminescence layer and a cathode.

FIG. 3 is a schematic sectional view of an element that is an embodiment of the present invention having an anode, a positive hole-transporting layer, a luminescence layer, an electron-transporting layer and a cathode.

FIG. 4 is a schematic sectional view of an element that is an embodiment of the present invention having an anode, a luminescence layer, an electron-transporting layer and a cathode.

### PREFERRED EMBODIMENT OF THE INVENTION

The first compound of the present invention is a compound represented by the formula (1)

In the formula (1), R₁₁ represents a non-substituted or substituted alkyl group, a non-substituted or substituted alkoxy group, a halogen atom, a cyano group, or a nitro group. The non-substituted or substituted alkyl group may be those having 1 to 12 carbon atoms. The non-substituted or substituted alkoxyl group may be those having 1 to 12 carbon atoms. Examples of the halogen atom may include —F, —Cl, —Br and —I. n₁₁ is an integer of 0 to 4. When n₁₁ is an integer of 2 or more, i.e. a plurality of R₁₁ exist, R₁₁, may be the same as or different from each other.

In the formula (1), (i) R₂₁ and R₃₁ are the same as or different from each other and each represents a hydrogen atom (provided that one of R₂₁ and R₃₁ is not hydrogen atom), a non-substituted or substituted alkyl group (provided that one of R₂₁ and R₃₁ is not the alkyl group), a non-substituted or substituted cycloalkyl group (provided that one of R₂₁ and R₃₁ is not the cycloalkyl group), a non-substituted or substituted aromatic group, or a non-substituted or substituted heteroaromatic group; or (ii) R₂₁ and R₃₁ together form a condensed ring consisting of non-substituted or substituted aromatic rings or non-substituted or substituted heteroaromatic rings. In the case of (i), it is preferable that at least one of R₂₁ and R₃₁ is a non-substituted or substituted aromatic group, or a non-substituted or substituted heteroaromatic group. The non-substituted or substituted alkyl group may preferably be those having 1 to 12 carbon atoms, and the non-substituted or substituted cycloalkyl group may preferably be those having 3 to 8 carbon atoms. Examples of the non-substituted or substituted aromatic group may include a phenyl group, a naphthyl group, a phenanthryl group, an anthryl group, pyrenyl group, and these groups substituted by one or more of a methyl group, a t-butyl group, a trifluoromethyl group, a halogen atom, a phenyl group, a methoxy group, a nitro group, a benzyl group, a cyclohexyl group and a cyano group. Examples of the non-substituted or substituted aromatic group may include a thienyl group, a pyridyl group and a quinolyl group. In the case of (ii), examples of the condensed ring that R₂₁ and R₃₁ together may form may include a dibenzocycloheptenylidene group, a dibenzocycloheptanylidene group, and a tribenzocycloheptatriene group.

The compound of the formula (1) according to the present invention may be produced by the four general synthesis routes, i.e., reaction of the compound represented by the formula (3) and the aldehyde derivative represented by the formula (4); reaction of the compound represented by the formula (5) and the compound represented by the formula (6); reaction of the dialdehyde represented by the formula (7) and the compound represented by the formula (8); and reaction of the compound represented by the formula (9) and the ketone derivative represented by the formula (10). In the formulae (3), (6), (8) and (9), Z represents —PO(OR)₂, or —PA₃⁺ or a salt thereof, wherein R represents a non-substituted or substituted alkyl group, and preferably a non-substituted or substituted alkyl group having 1 to 4 carbon atoms. “A” of —PA₃⁺ represents a non-substituted or substituted aryl group, and preferably a phenyl group, a tolyl group or a naphthyl group. When two or more Z's are exist in the formula, these maybe the same as or different from each other. The salt of —PA₃⁺ may be those in which —PA₃⁺ and any of suitable base are combined. Examples of the base may include ions of halogen atoms such as fluorine, chlorine, bromine and iodine.

All of these reactions are those between an aldehyde or ketone and an active methylene, and are usually performed in a solvent such as an organic solvent with a base. Examples of the solvent for reaction may include water; alcohols such as methanol, ethanol, butanol and amyl alcohol; aromatic hydrocarbons such as benzene, toluene, xylene, mesitylene, chlorobenzene and nitrobenzene; ethers such as diethylether, tetrahydrofuran and dioxane; halogenated hydrocarbons such as chloroform, dichlormethane and dichloroethane; heterocyclic aromatic hydrocarbons such as pyridine and quinoline; and other organic solvents such as N,N-dimethylformamide and dimethylsulfoxide. Any of generally used organic solvents may be used. Examples of the bases for reaction may include inorganic bases such as potassium carbonate, sodium carbonate, potassium hydroxide and sodium hydroxide; organic bases such as triethylamine, triethanolamine, pyridine and hexamethylenetetramine; alkali metal salts of alcohols such as sodiummethoxide, sodium ethoxide and potassium butoxide; and sodium amides. The amount of the base may suitably adjusted from catalytic amount to chemical equivalent amount.

The temperature for reaction may be from about −10° C. to about 150° C., and preferably from about 0° C. to about 80° C. in most of cases. The reaction time depends upon the reaction temperature, and may usually be 30 minutes to 100 hours, which may suitably be adjusted depending on the combination of the reaction materials.

There is no particular limitation as to the operation for separating the objective compound from the reaction mixture after finishing the reaction. For example, a crude product may be retrieved by concentration or dilution with a poor solvent, followed by removal of inorganic matters preferably by washing with water, and then any general purification procedures such as column chromatography, recrystalization, or sublimation purification, for obtaining a pure product.

The second compound of the present invention is the compound represented by the formula (2).

The compound represented by the formula (2) maybe obtained by the reaction between the compound represented by the formula (3) and the tolualdehyde derivative represented by the formula (11), or the reaction between the dialdehyde represented by the formula (7) and the compound represented by the formula (12). In the formula (12), Z represents —PO(OR)₂ or —PA₃⁺ or a salt thereof, wherein R represents a non-substituted or substituted alkyl group, and preferably a non-substituted or substituted alkyl group having 1 to 4 carbon atoms. “A” represents a non-substituted or substituted aryl group, and preferably a phenyl group, a tolyl group or a naphthyl group. The salt of —PA₃⁺ may be those in which —PA₃⁺ and any of suitable base are combined. Examples of the base may include ions of halogen atoms such as fluorine, chlorine, bromine and iodine.

Similar to the reactions for synthesizing the compound represented by the formula (1), the reactions for synthesizing the compound represented by the formula (2) are those between an aldehyde and an active methylene, and are performed in a solvent such as an organic solvent with a base. The solvents and bases for reaction as well as the reaction conditions may be the same as those described in the explanation of the synthetic routes of the compound represented by the formula (1).

The compound represented by the formula (2) may be used as an intermediate for obtaining the compound of the formula (9) that is a useful material for obtaining the compound of the formula (1). That is, the compound represented by the formula (2) may be modified to be a halomethyl derivative represented by the following formula (13): 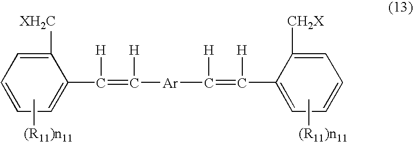
wherein R₁₁ and n₁₁ are the same as those in the formula (1), and X represents a halogen. The compound represented by the formula (9) may be produced by the subsequent reaction with a trialkyl phosphite, or reaction with a triaryl phosphine compound such as triphenyl phosphine.

The reaction of the compound of the formula (2) to the halomethyl derivative of the formula (13) may be readily performed by the reaction of a compound containing a halogen species and the compound represented by the formula (2), under the light irradiation and/or in the presence of a radical generator such as benzoyl peroxide or azobisisobutyronitrile, in an organic solvent such as carbon tetrachloride or carbon disulfide at about 30° C. to 100° C. for 30 minutes to 10 hours. The compound containing the halogen species may preferably be a compound containing a bromine atom, such as bromine or N-bromosuccinimide. Examples of the methods for converting the resulting halomethyl derivative of the formula (13) into the compound of the formula (9) may include a method in which the halomethyl derivative is reacted with a phosphite triester at 50° C. to 150° C. for 10 minutes to 10 hours to obtain a bismethyl phosphonate derivative; and a method in which the halomethyl derivative is reacted with a triaryl phosphine compound such as triphenyl phosphine to obtain a bismethyl triaryl phosphonium compound.

Embodiments of the compound represented by the formula (1) of the present invention will be described in Tables 1 to 36, and embodiments of the compound represented by the formula (2) in Tables 37 to 40, although the present invention is not limited thereto.

TABLE 1

| Compound No. | Formula |
|---|---|
| 1-01 | 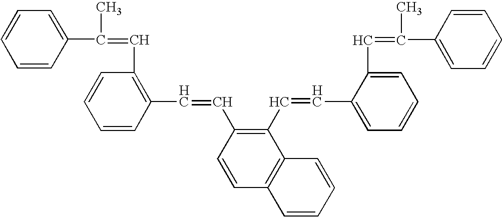 |
| 1-02 | 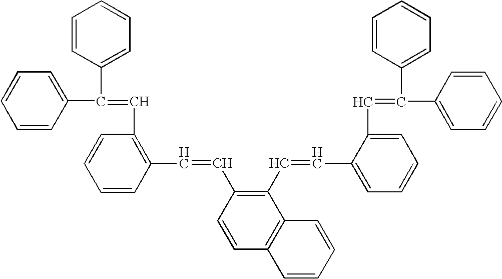 |
| 1-03 | 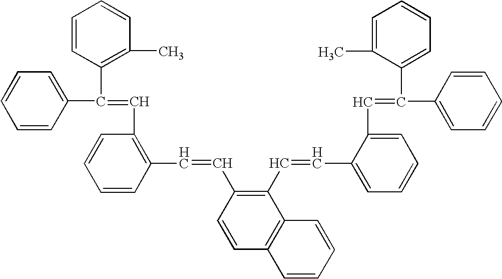 |
| 1-04 | 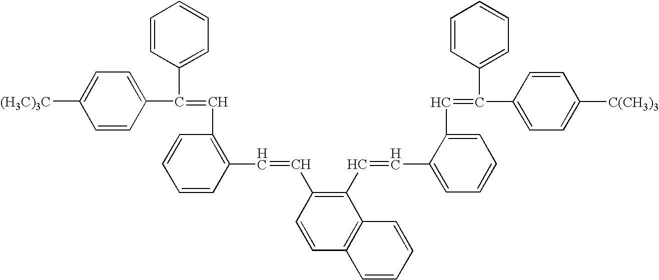 |

TABLE 2

| Compound No. | Formula |
|---|---|
| 1-05 | 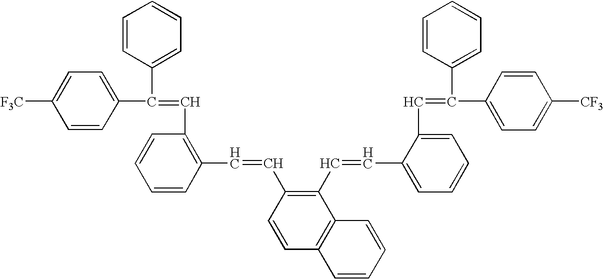 |
| 1-06 | 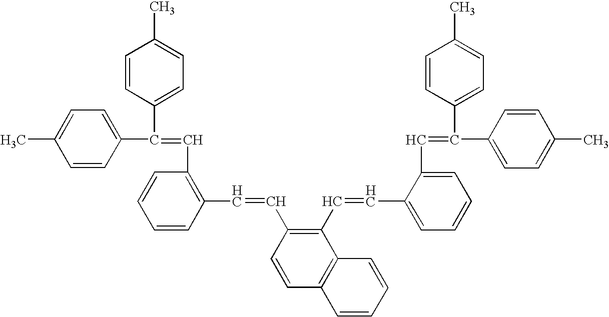 |
| 1-07 | 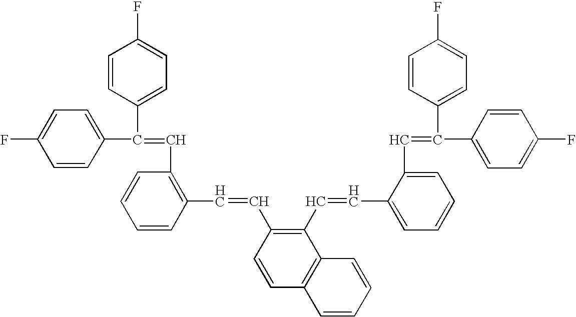 |
| 1-08 | 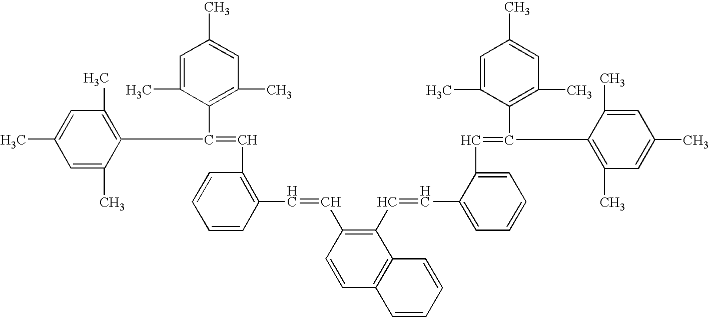 |

TABLE 3

| Compound No. | Formula |
|---|---|
| 1-09 | 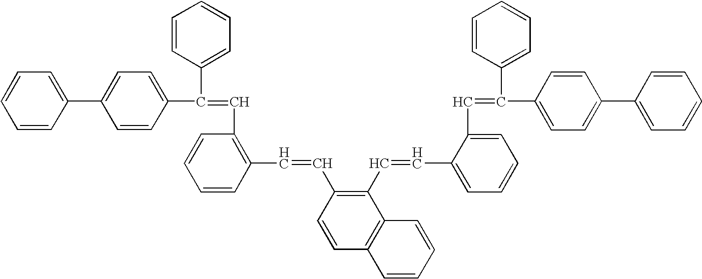 |
| 1-10 | 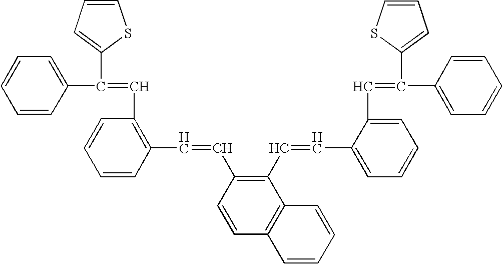 |
| 1-11 | 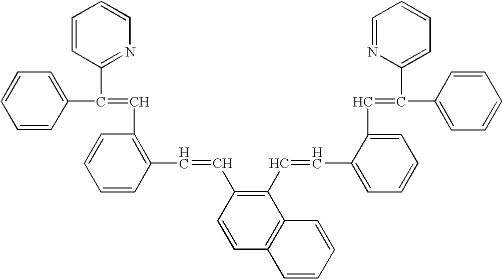 |
| 1-12 | 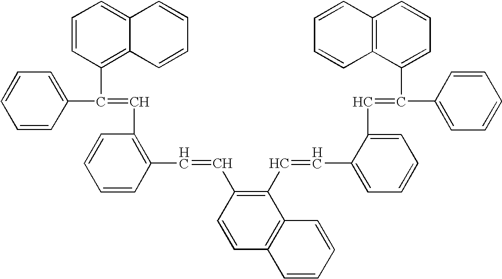 |

TABLE 4

| Compound No. | Formula |
|---|---|
| 1-13 | 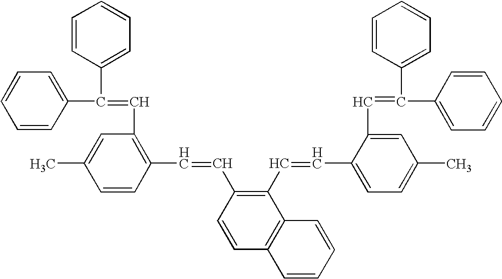 |
| 1-14 | 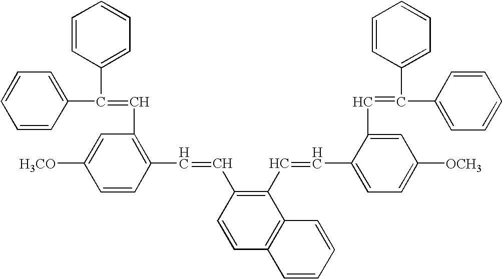 |
| 1-15 | 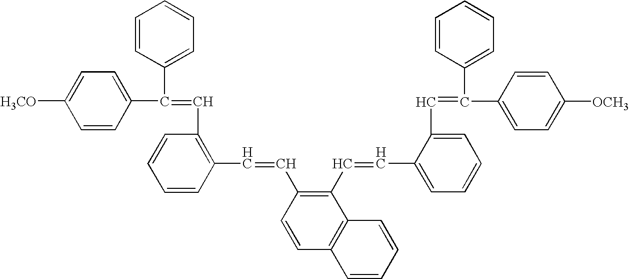 |
| 1-16 | 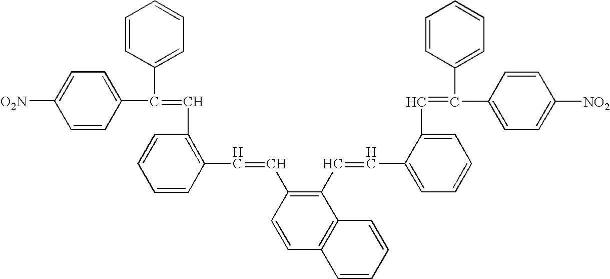 |

TABLE 5

| Compound No. | Formula |
|---|---|
| 1-17 | 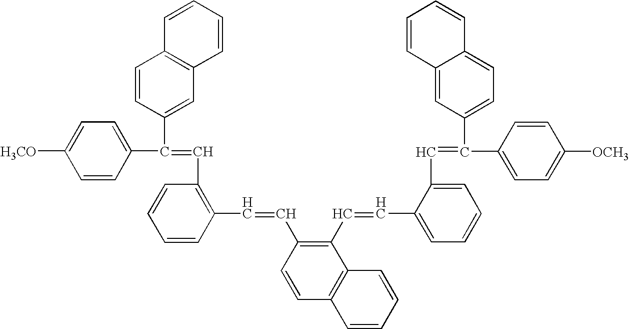 |
| 1-18 | 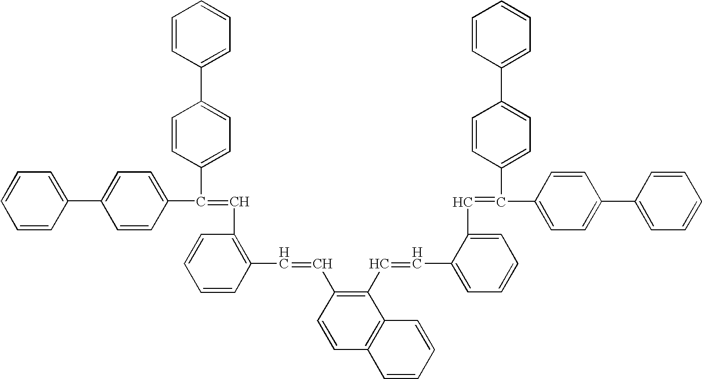 |
| 1-19 | 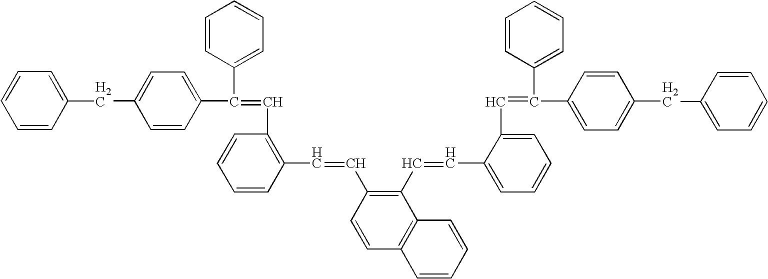 |
| 1-20 | 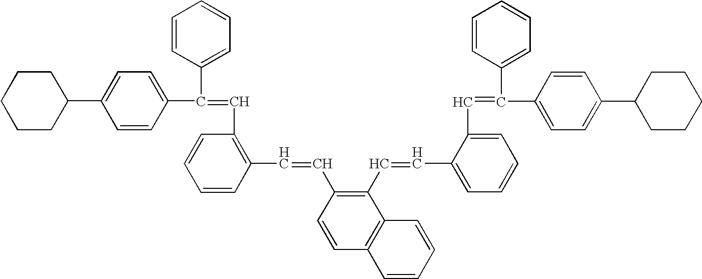 |

TABLE 6

| Compound No. | Formula |
|---|---|
| 1-21 | 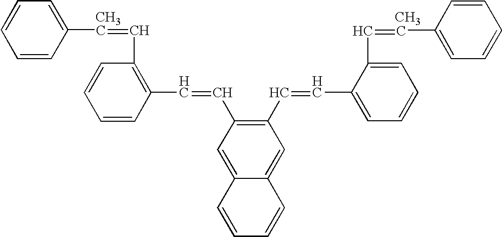 |
| 1-22 | 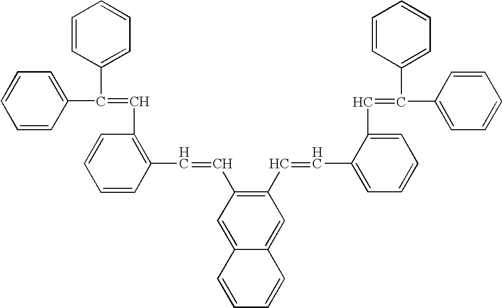 |
| 1-23 | 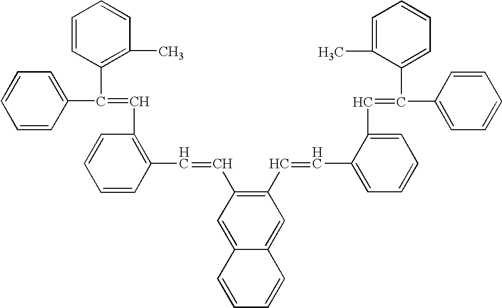 |
| 1-24 | 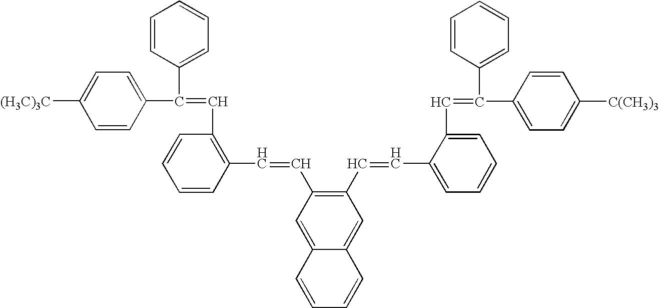 |

TABLE 7

| Compound No. | Formula |
|---|---|
| 1-25 | 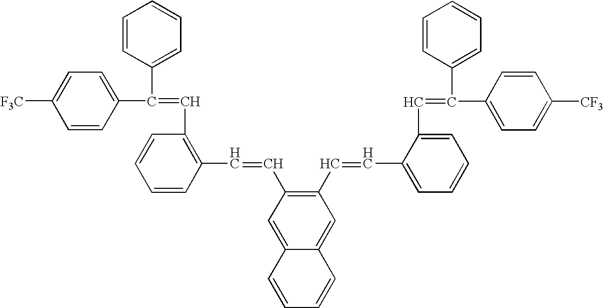 |
| 1-26 | 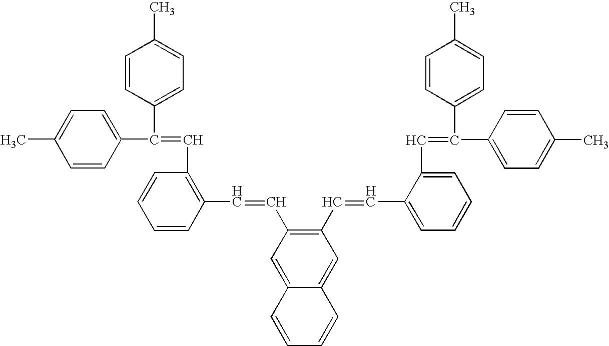 |
| 1-27 | 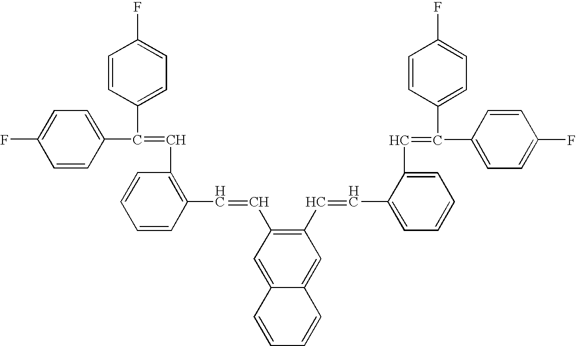 |
| 1-28 | 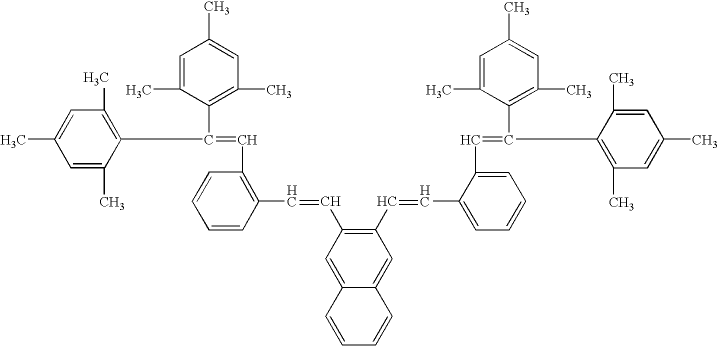 |

TABLE 8

| Compound No. | Formula |
|---|---|
| 1-29 | 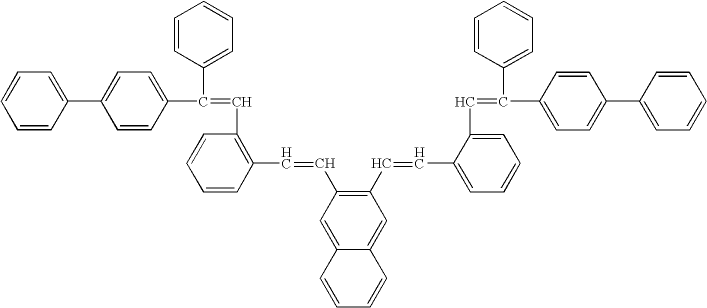 |
| 1-30 | 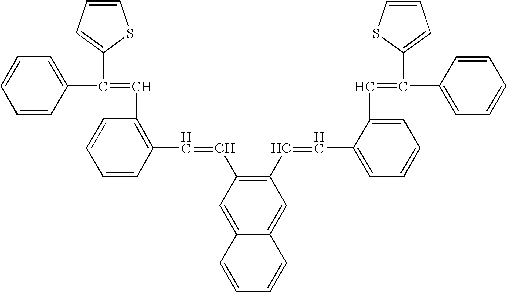 |
| 1-31 | 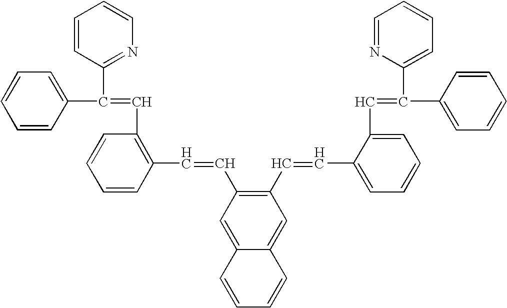 |
| 1-32 | 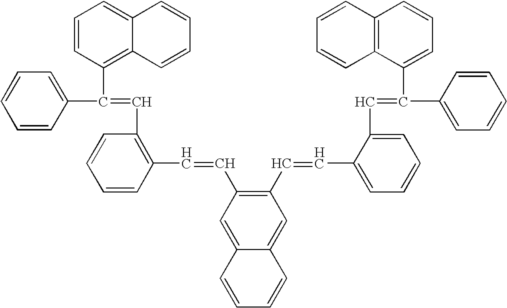 |

TABLE 9

| Compound No. | Formula |
|---|---|
| 1-33 | 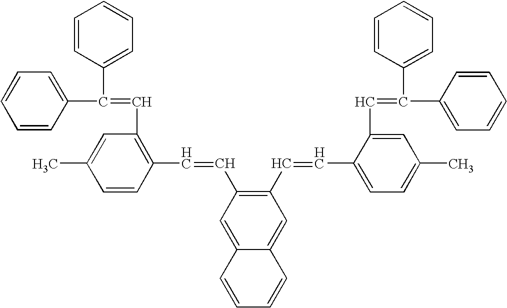 |
| 1-34 | 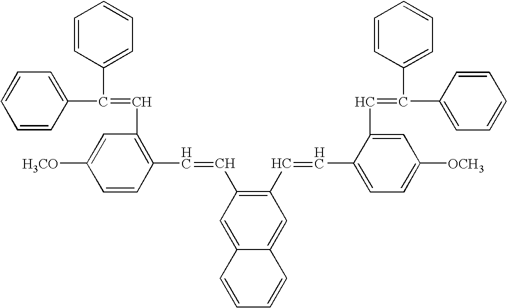 |
| 1-35 | 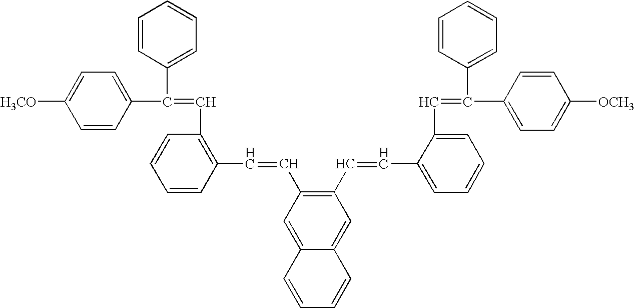 |
| 1-36 | 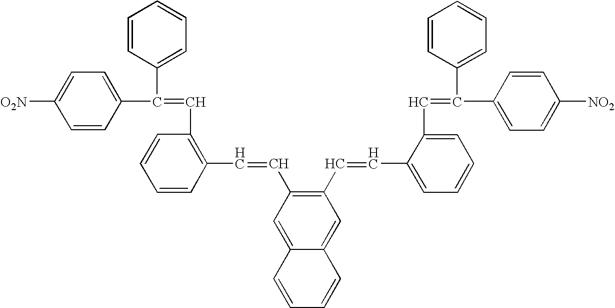 |

TABLE 10

| Compound No. | Formula |
|---|---|
| 1-37 | 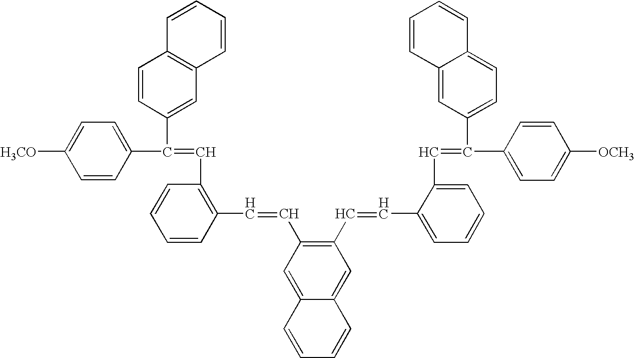 |
| 1-38 | 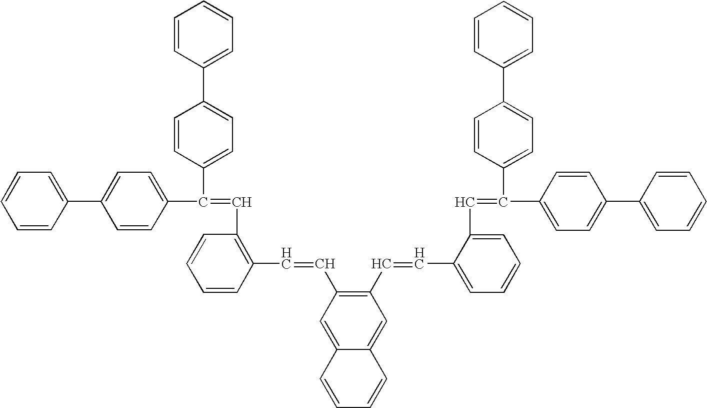 |
| 1-39 | 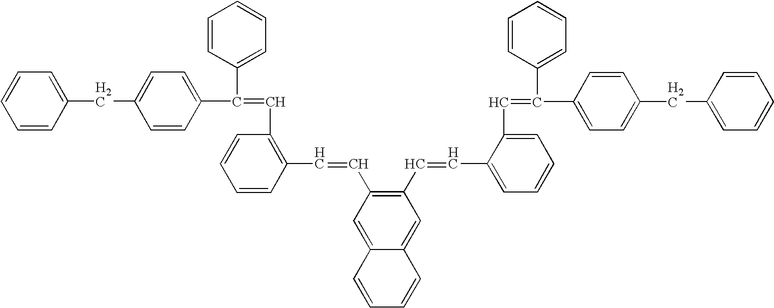 |
| 1-40 | 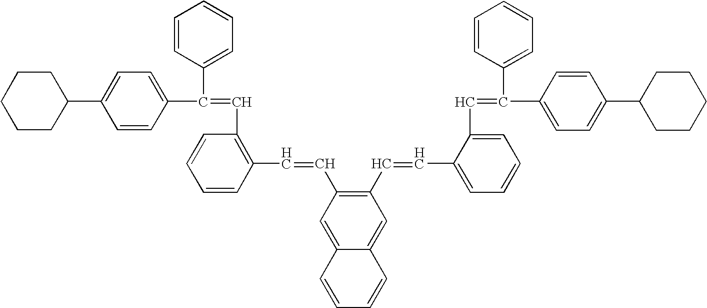 |

TABLE 11

| Compound No. | Formula |
|---|---|
| 1-41 | 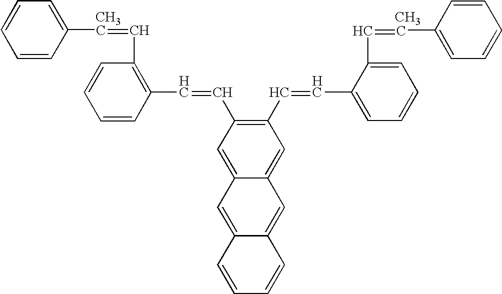 |
| 1-42 | 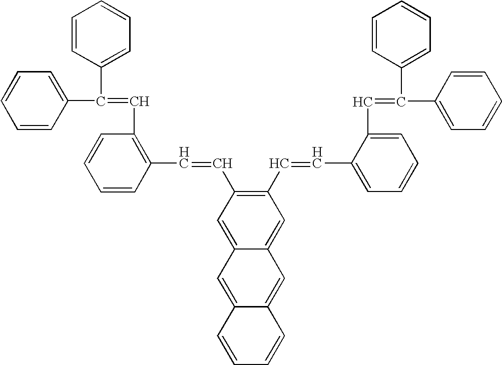 |
| 1-43 | 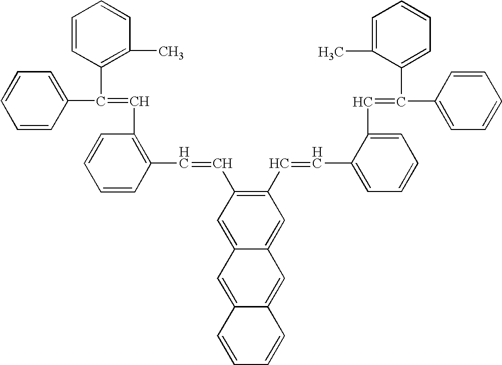 |
| 1-44 | 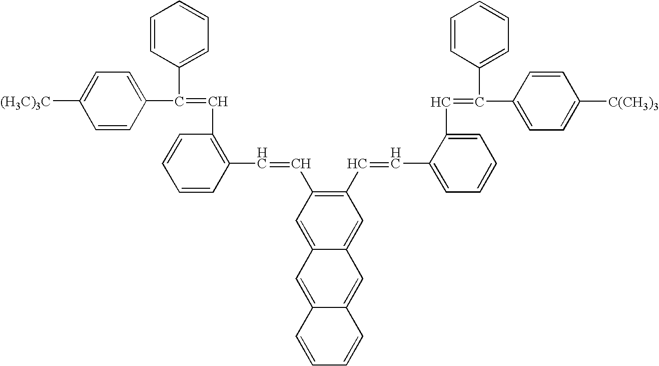 |

TABLE 12

| Compound No. | Formula |
|---|---|
| 1-45 | 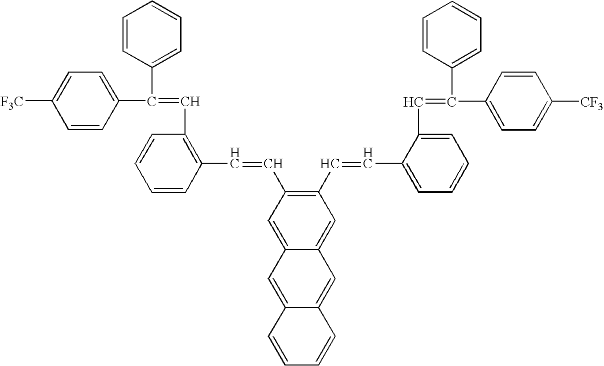 |
| 1-46 | 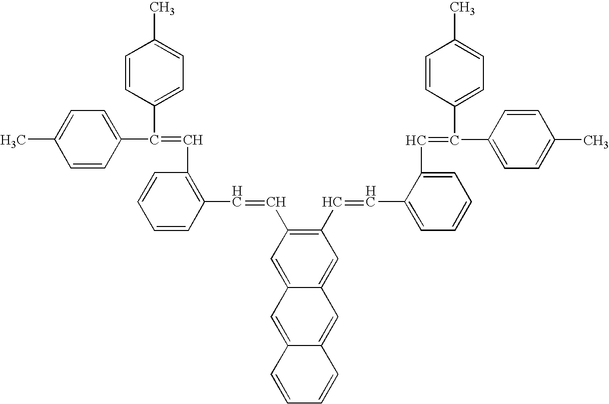 |
| 1-47 | 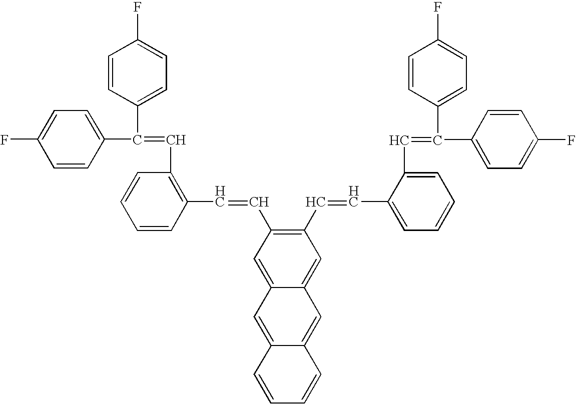 |
| 1-48 | 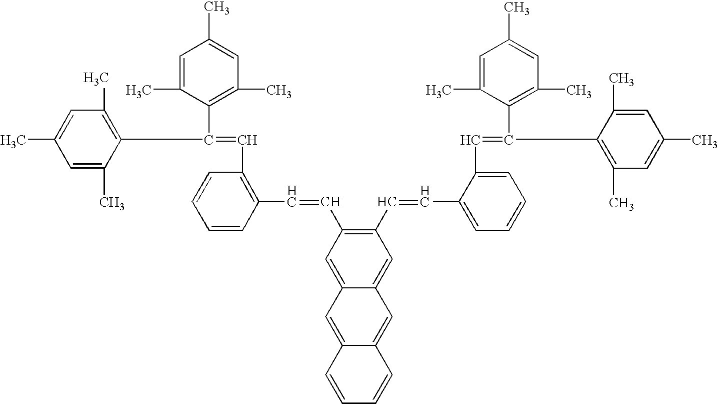 |

TABLE 13

| Compound No. | Formula |
|---|---|
| 1-49 | 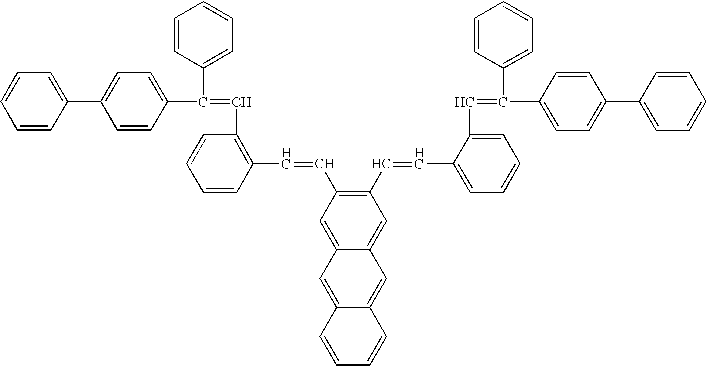 |
| 1-50 | 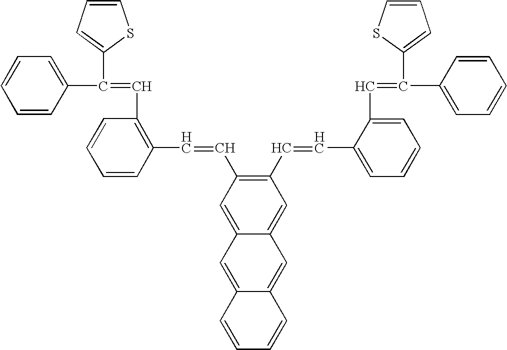 |
| 1-51 | 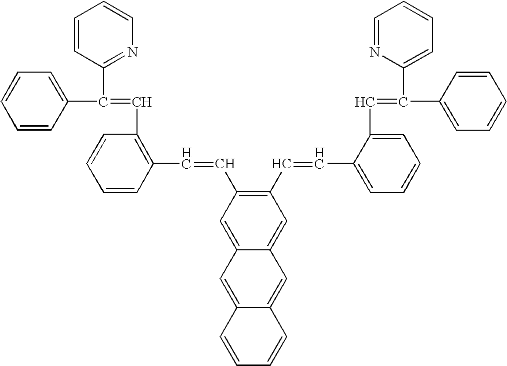 |
| 1-52 | 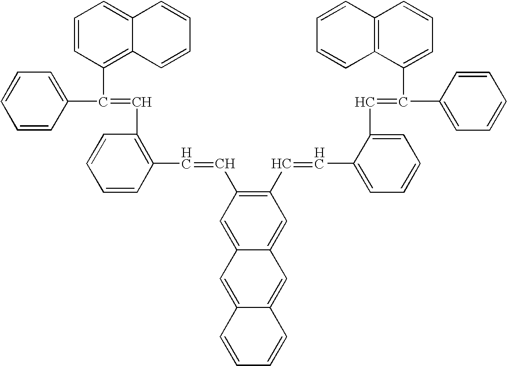 |

TABLE 14

| Compound No. | Formula |
|---|---|
| 1-53 | 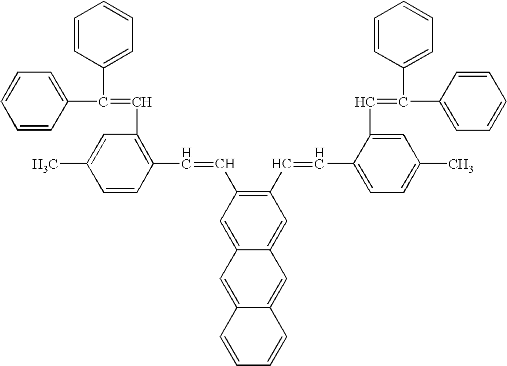 |
| 1-54 | 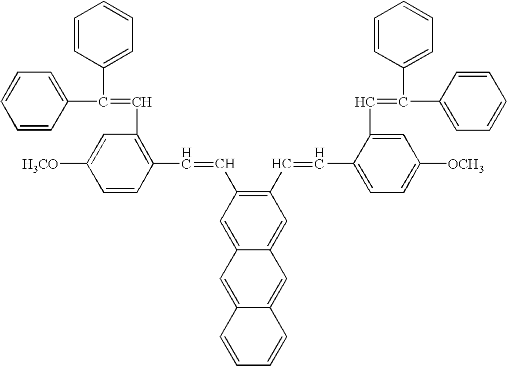 |
| 1-55 | 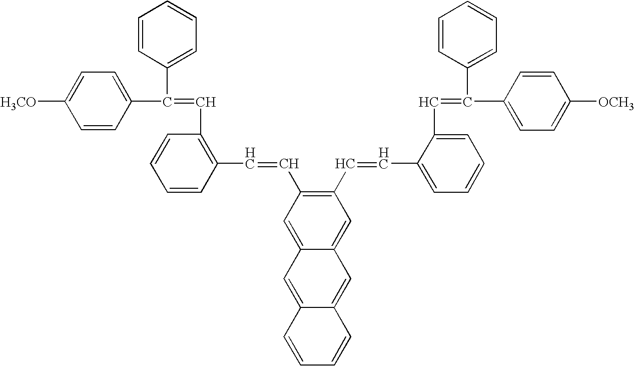 |
| 1-56 | 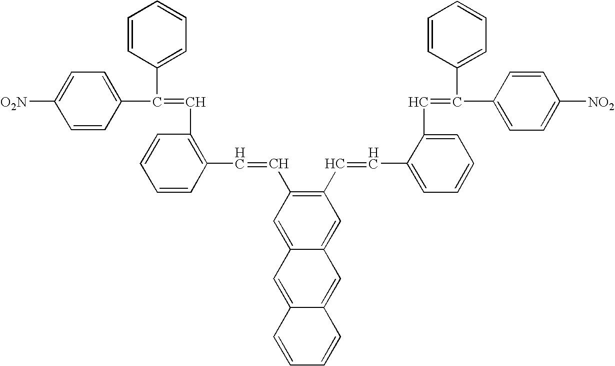 |

TABLE 15

| Compound No. | Formula |
|---|---|
| 1-57 | 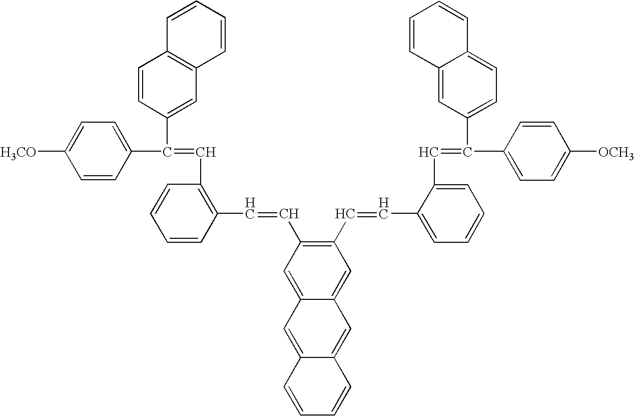 |
| 1-58 | 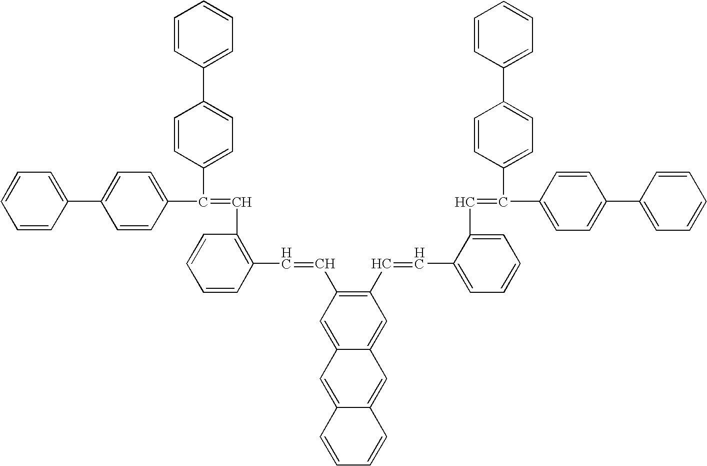 |
| 1-59 | 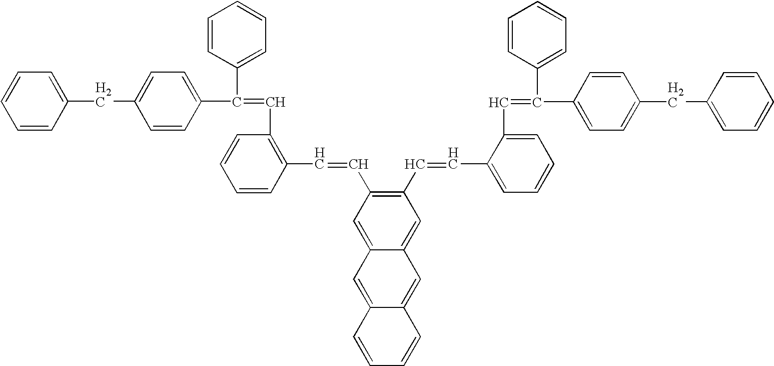 |
| 1-60 | 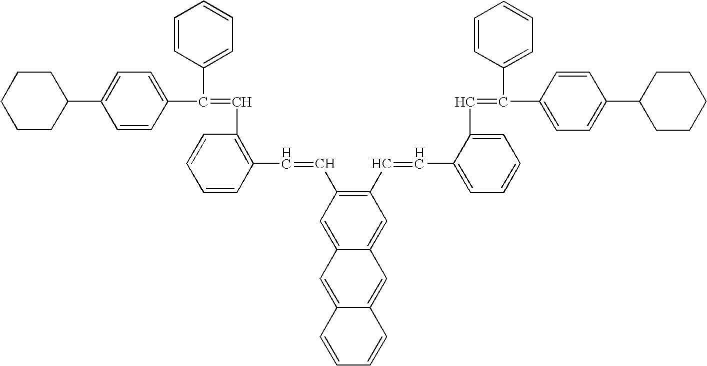 |

TABLE 16

| Compound No. | Formula |
|---|---|
| 1-61 | 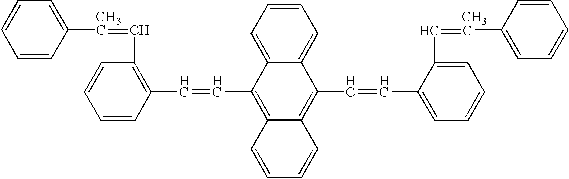 |
| 1-62 | 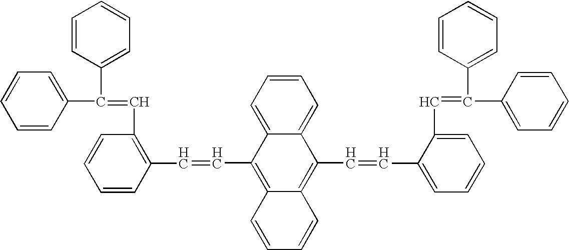 |
| 1-63 | 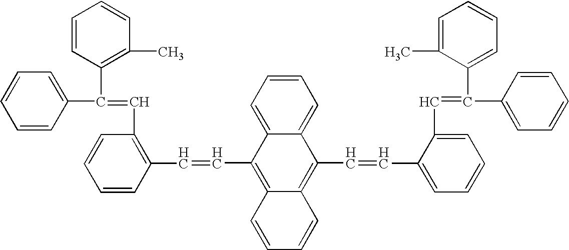 |
| 1-64 | 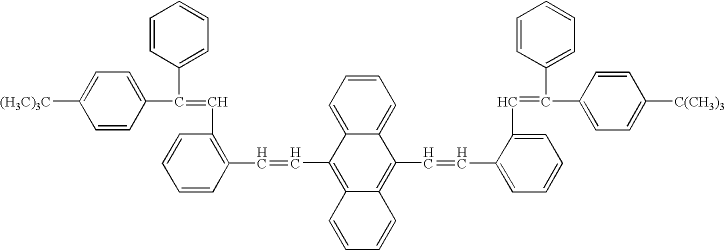 |

TABLE 17

| Compound No. | Formula |
|---|---|
| 1-65 | 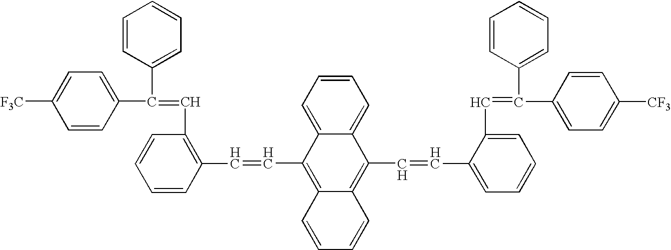 |
| 1-66 | 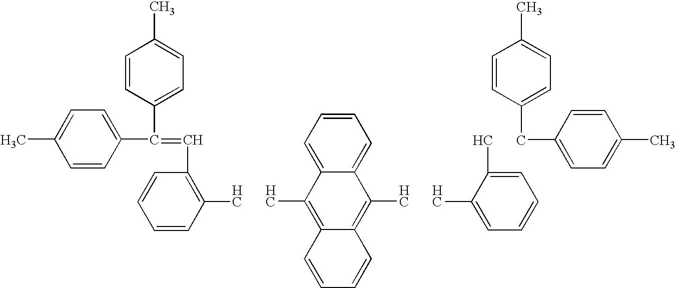 |
| 1-67 | 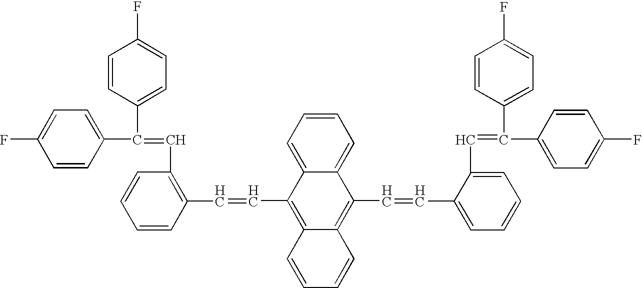 |
| 1-68 | 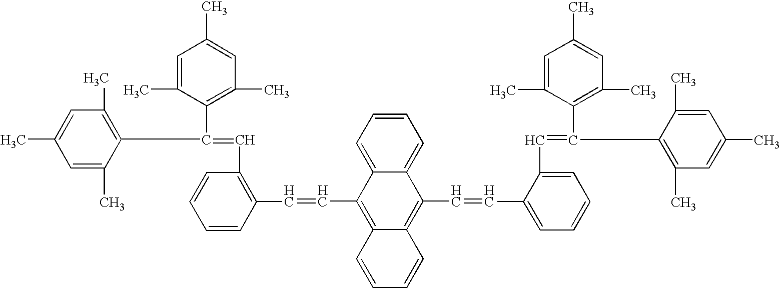 |

TABLE 18

| Compound No. | Formula |
|---|---|
| 1-69 | 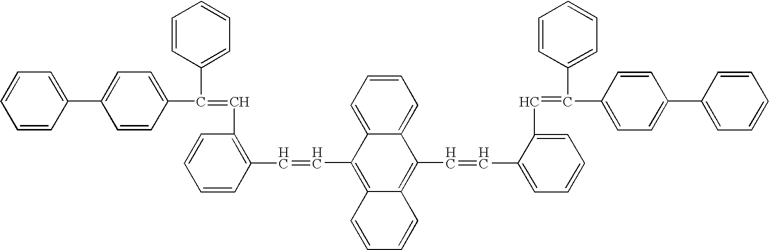 |
| 1-70 | 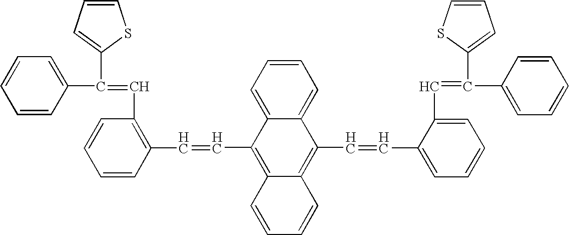 |
| 1-71 | 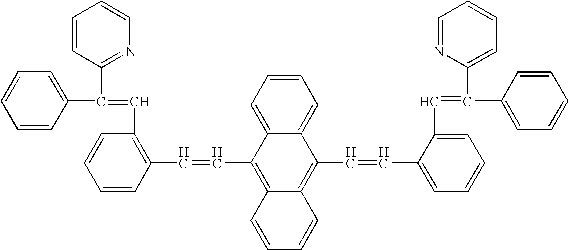 |
| 1-72 | 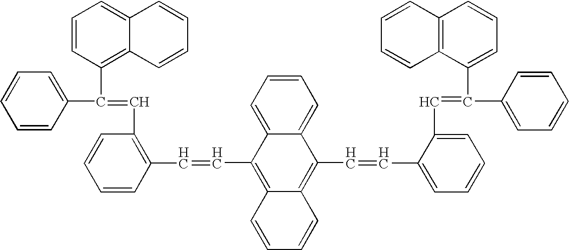 |

TABLE 19

| Compound No. | Formula |
|---|---|
| 1-73 | 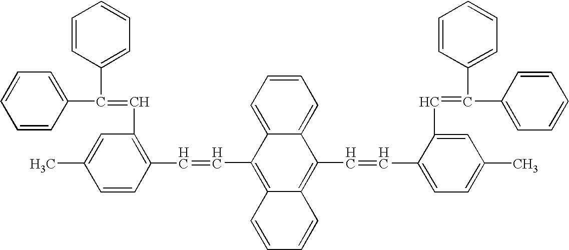 |
| 1-74 | 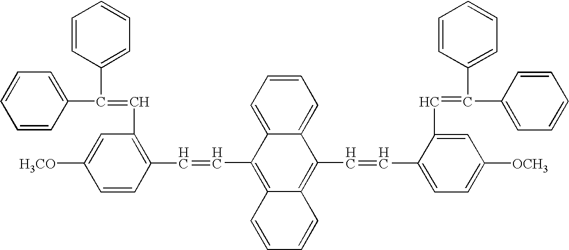 |
| 1-75 | 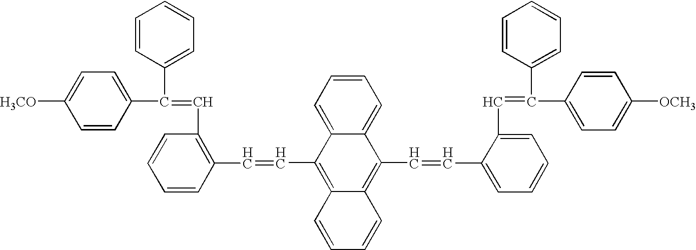 |
| 1-76 | 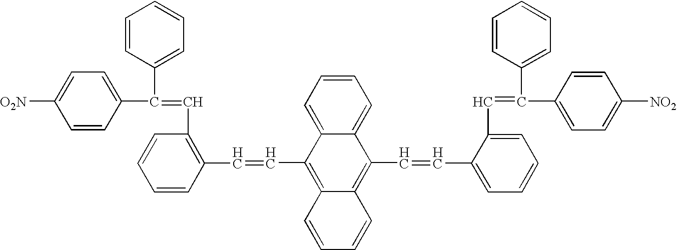 |

TABLE 20

| Compound | |
|---|---|
| No. | Formula |
| 1-77 | 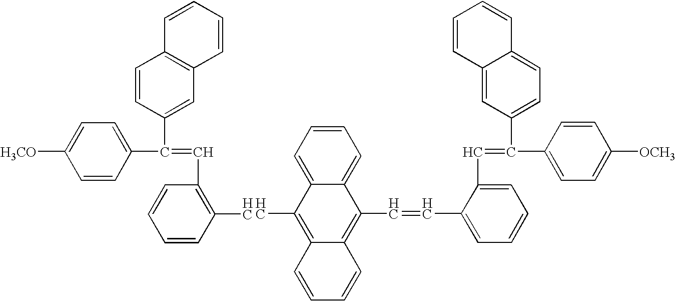 |
| 1-78 | 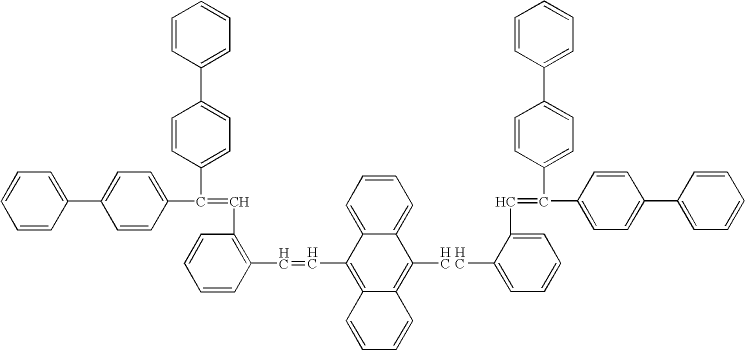 |
| 1-79 | 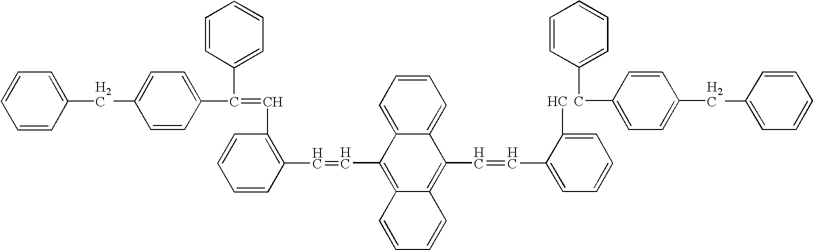 |
| 1-80 | 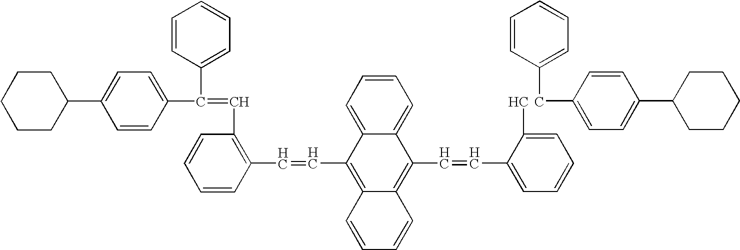 |

TABLE 21

| Compound | |
|---|---|
| No. | Formula |
| 1-81 | 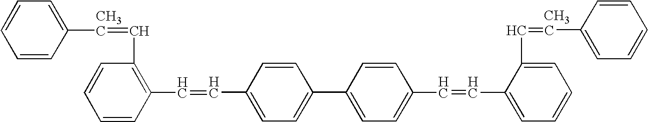 |
| 1-82 | 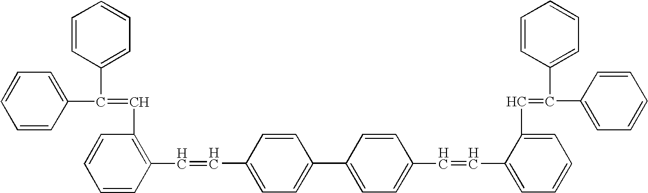 |
| 1-83 | 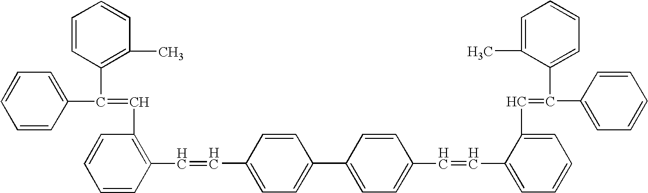 |
| 1-84 | 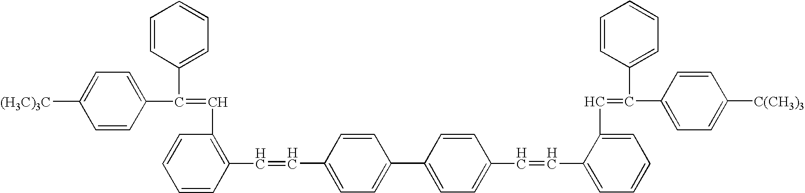 |

TABLE 22

| Compound | |
|---|---|
| No. | Formula |
| 1-85 | 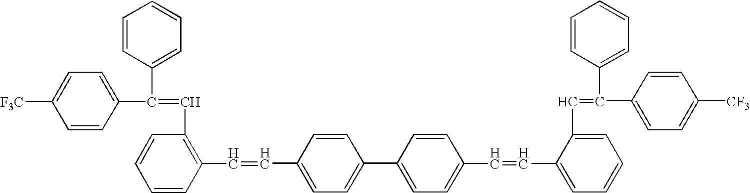 |
| 1-86 | 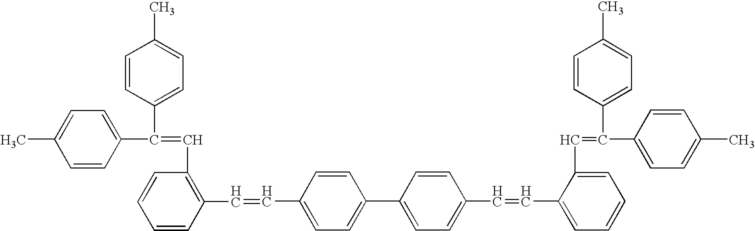 |
| 1-87 | 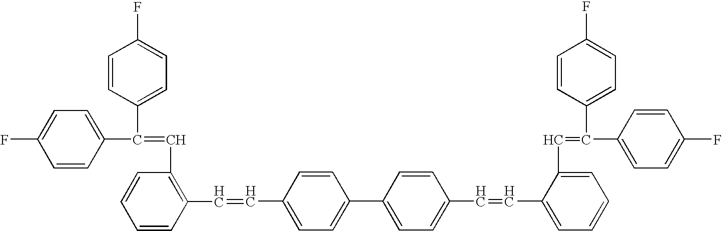 |
| 1-88 | 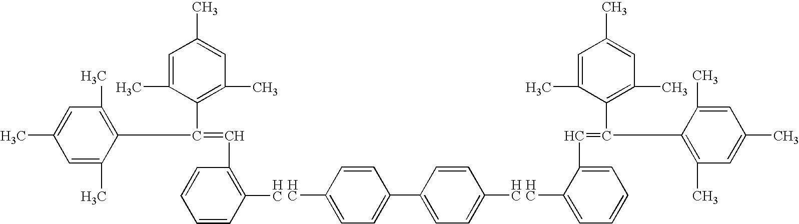 |

TABLE 23

| Compound | |
|---|---|
| No. | Formula |
| 1-89 | 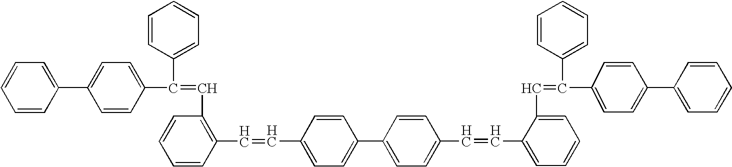 |
| 1-90 | 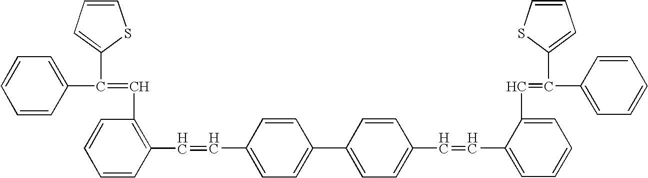 |
| 1-91 | 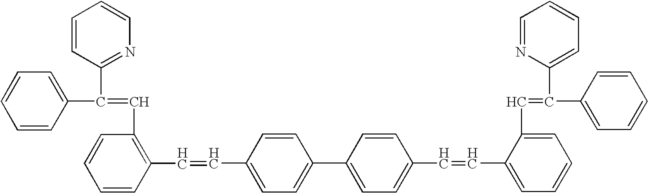 |
| 1-92 | 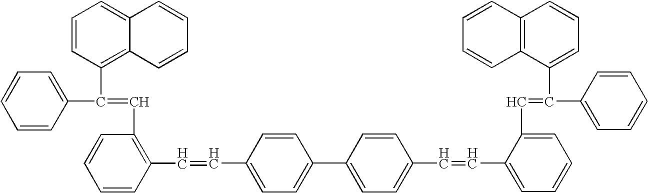 |

TABLE 24

| Compound | |
|---|---|
| No. | Formula |
| 1-93 | 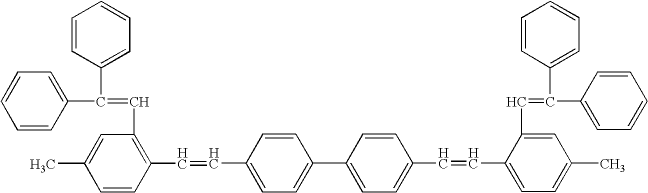 |
| 1-94 | 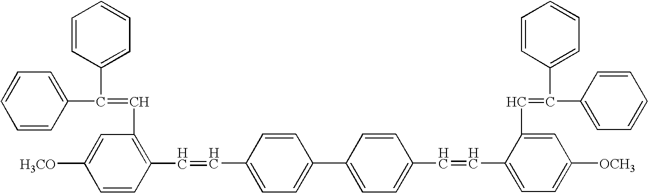 |
| 1-95 | 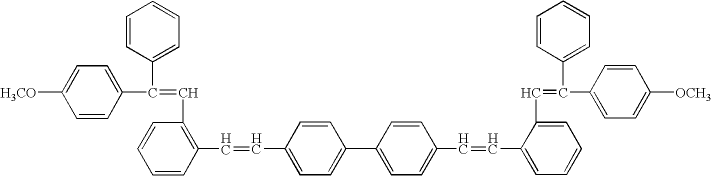 |
| 1-96 | 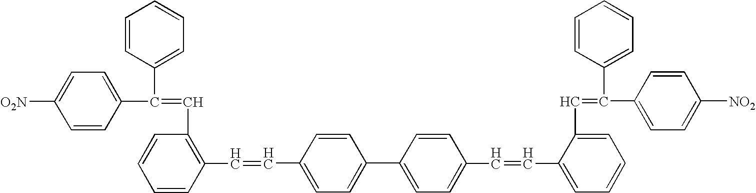 |

TABLE 25

| Compound | |
|---|---|
| No. | Formula |
| 1-97 | 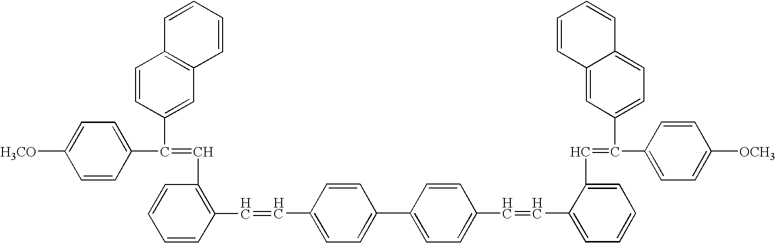 |
| 1-98 | 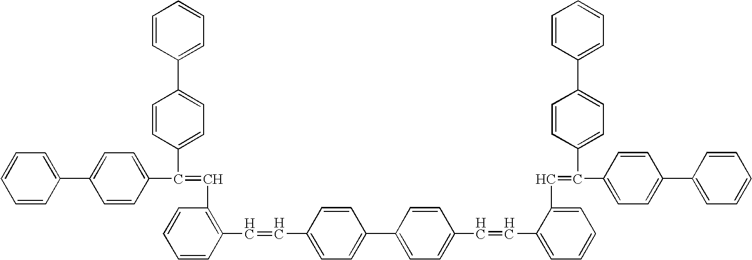 |
| 1-99 | 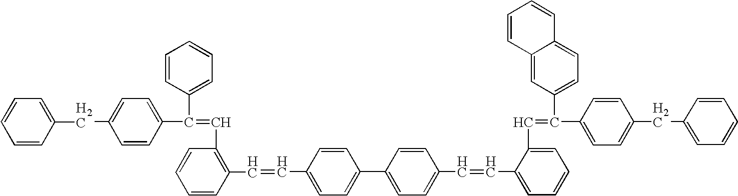 |
| 1-100 | 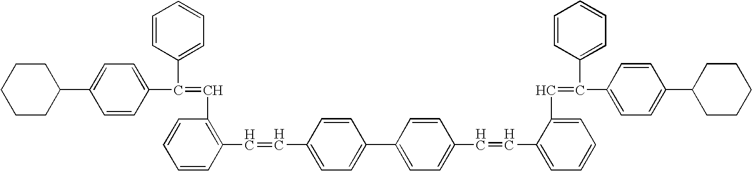 |

TABLE 26

| Compound | |
|---|---|
| No. | Formula |
| 1-101 | 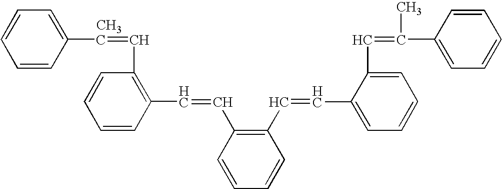 |
| 1-102 | 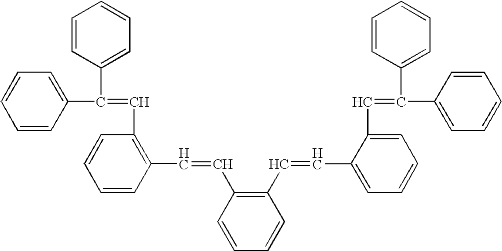 |
| 1-103 | 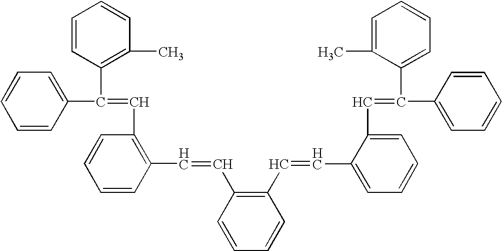 |
| 1-104 | 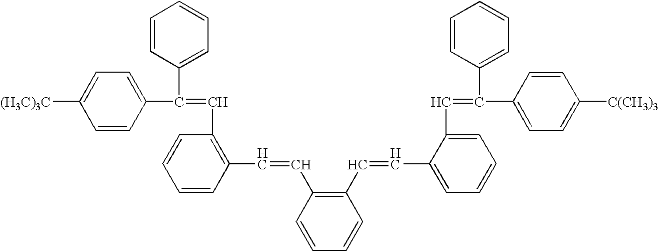 |

TABLE 27

| Compound | |
|---|---|
| No. | Formula |
| 1-105 | 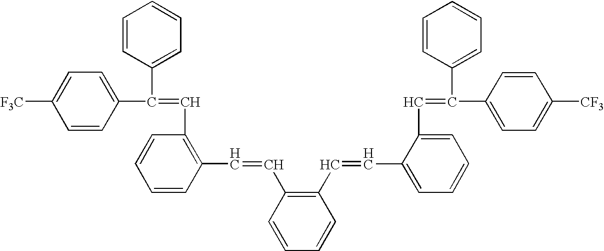 |
| 1-106 | 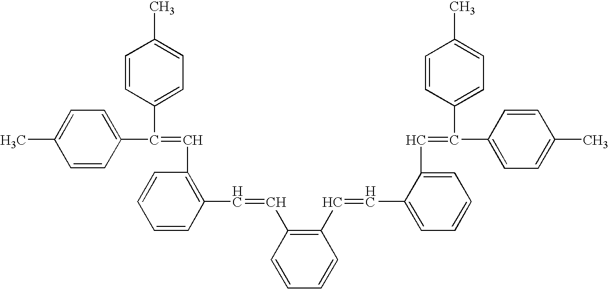 |
| 1-107 | 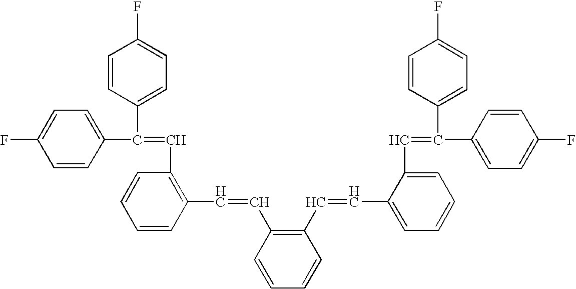 |
| 1-108 | 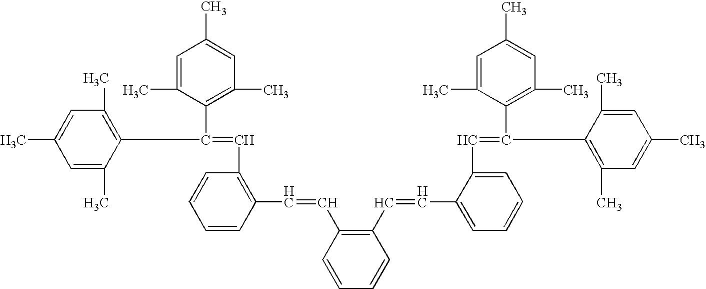 |

TABLE 28

| Compound | |
|---|---|
| No. | Formula |
| 1-109 | 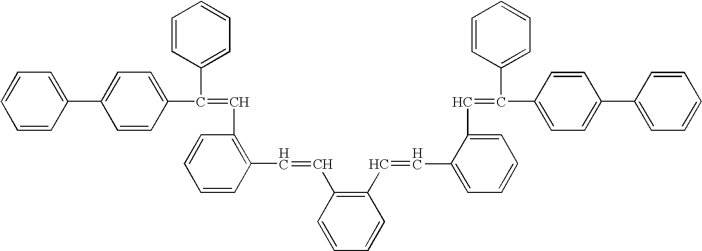 |
| 1-110 | 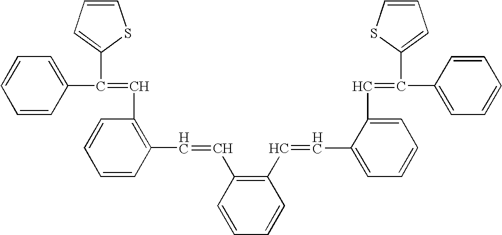 |
| 1-111 | 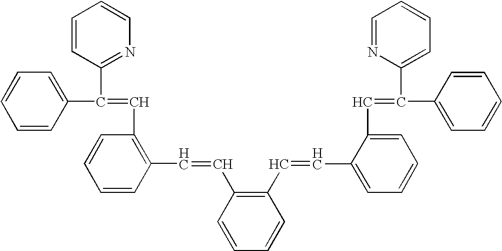 |
| 1-112 | 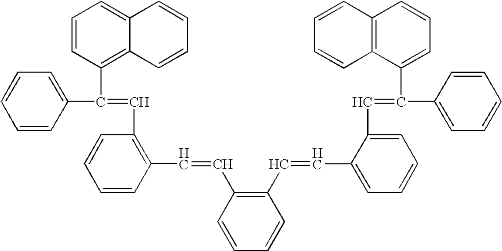 |

TABLE 29

| Compound | |
|---|---|
| No. | Formula |
| 1-113 | 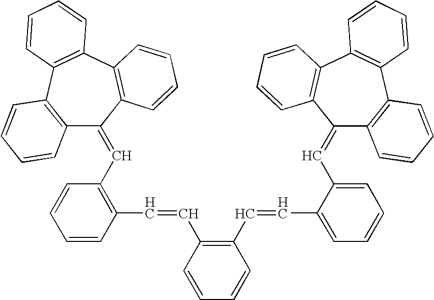 |
| 1-114 | 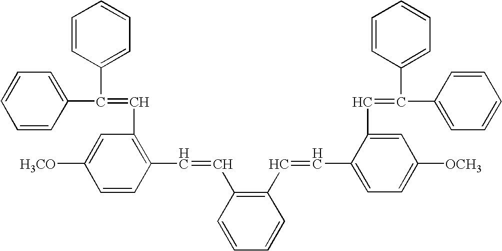 |
| 1-115 | 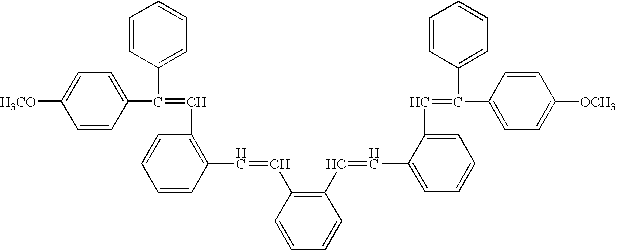 |
| 1-116 | 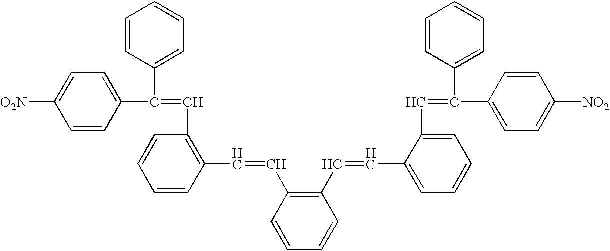 |

TABLE 30

| Compound | |
|---|---|
| No. | Formula |
| 1-117 | 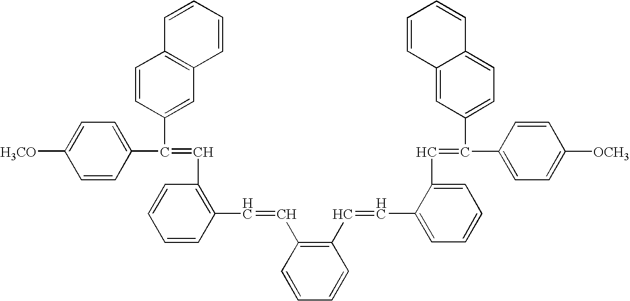 |
| 1-118 | 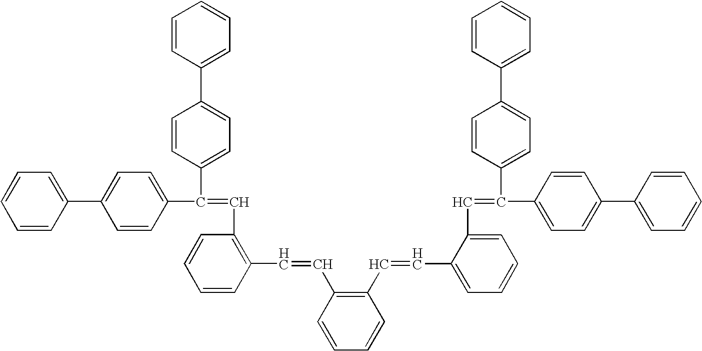 |
| 1-119 | 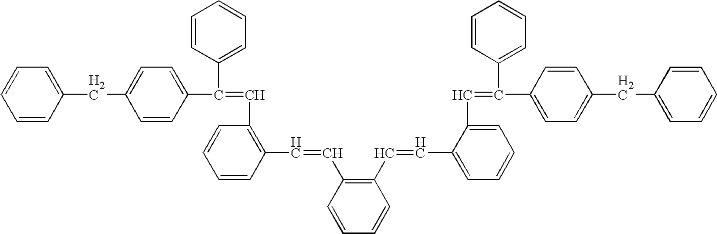 |
| 1-120 | 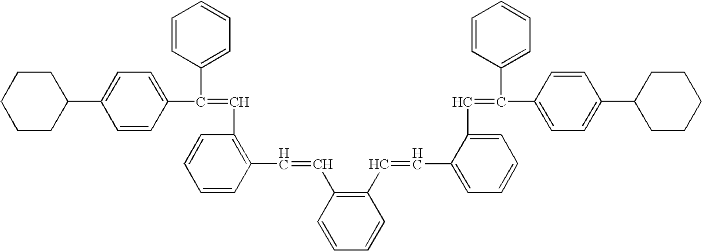 |

TABLE 31

| Compound | |
|---|---|
| No. | Formula |
| 1-121 | 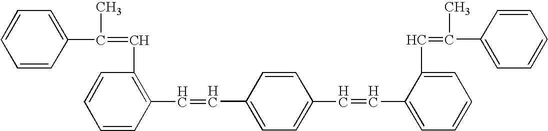 |
| 1-122 | 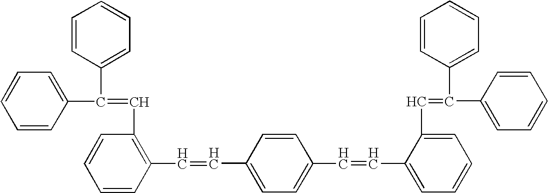 |
| 1-123 | 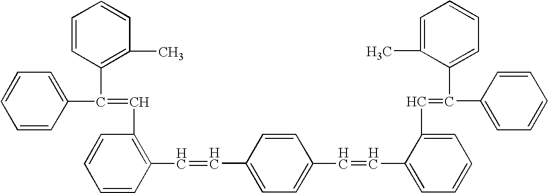 |
| 1-124 | 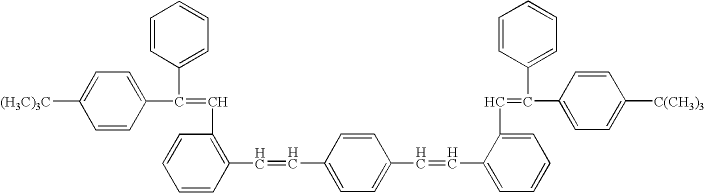 |

TABLE 32

| Compound | |
|---|---|
| No. | Formula |
| 1-125 | 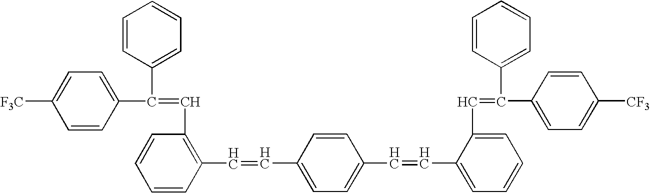 |
| 1-126 | 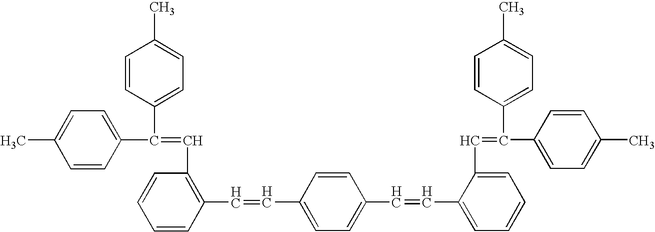 |
| 1-127 | 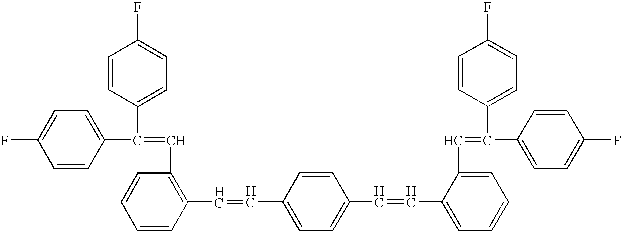 |
| 1-128 | 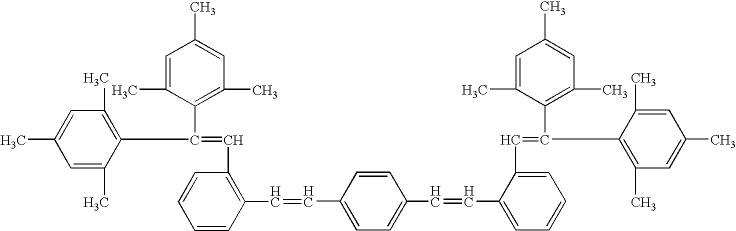 |

TABLE 33

| Compound | |
|---|---|
| No. | Formula |
| 1-129 | 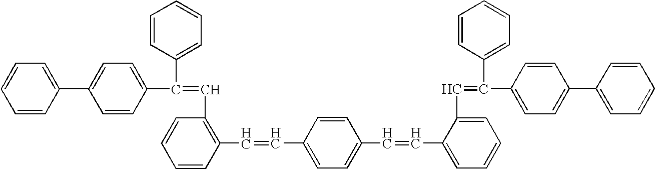 |
| 1-130 | 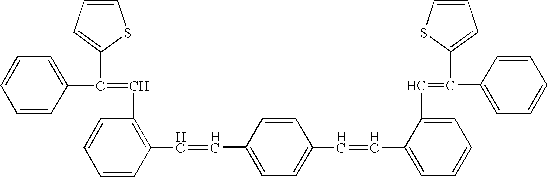 |
| 1-131 | 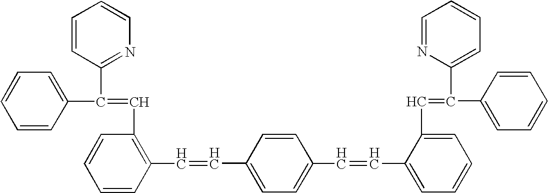 |
| 1-132 | 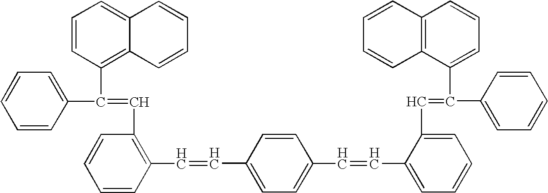 |

TABLE 34

| Compound | |
|---|---|
| No. | Formula |
| 1-133 | 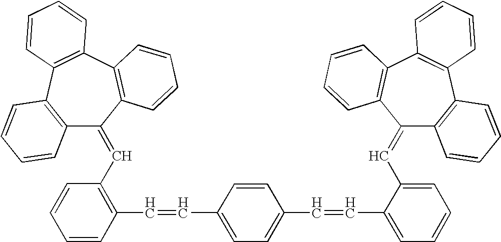 |
| 1-134 | 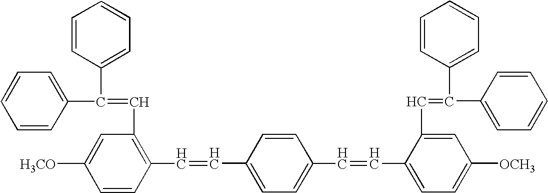 |
| 1-135 | 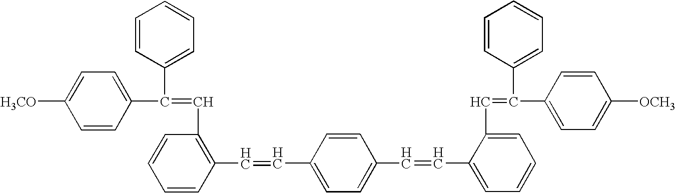 |
| 1-136 | 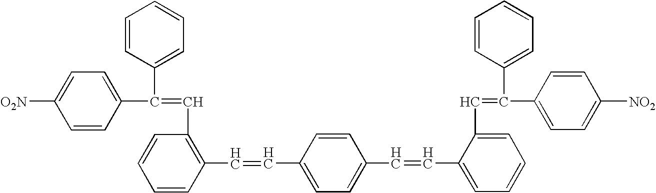 |

TABLE 35

| Compound | |
|---|---|
| No | Formula |
| 1-137 | 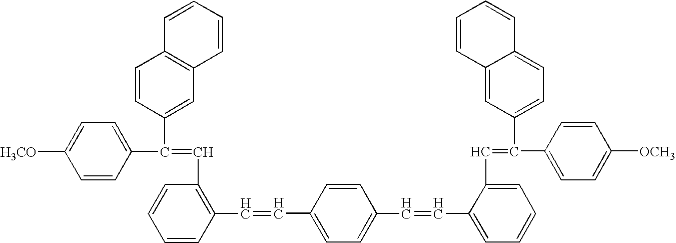 |
| 1-138 | 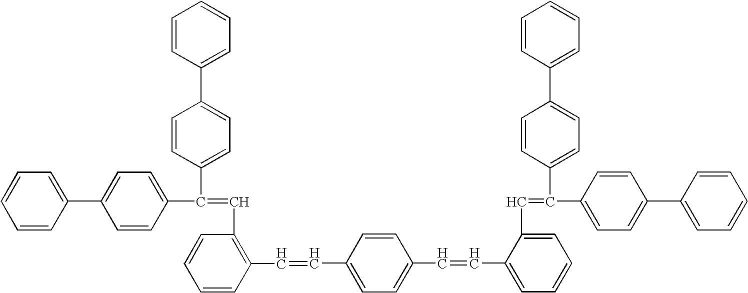 |
| 1-139 | 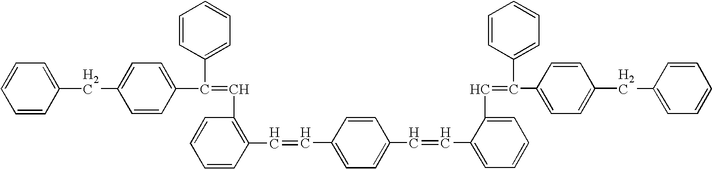 |
| 1-140 | 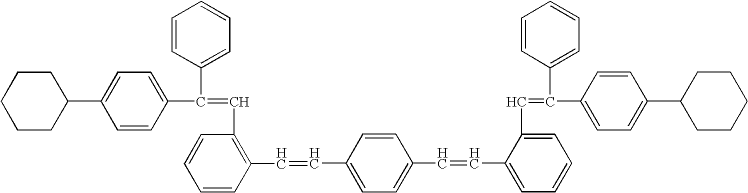 |

TABLE 36

| Compound | |
|---|---|
| No. | Formula |
| 2-01 | 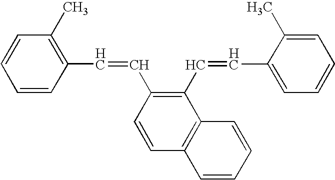 |
| 2-02 | 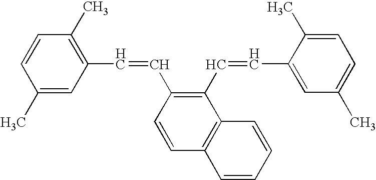 |
| 2-03 | 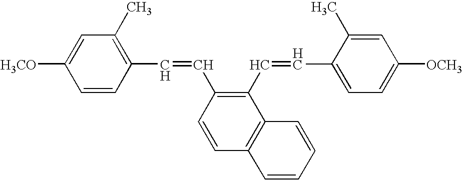 |
| 2-04 | 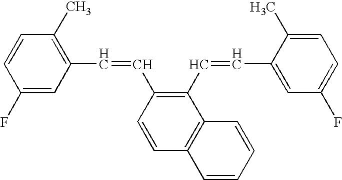 |

TABLE 37

| Compound | |
|---|---|
| No. | Formula |
| 2-05 | 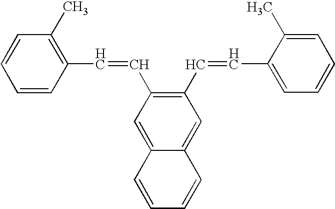 |
| 2-06 | 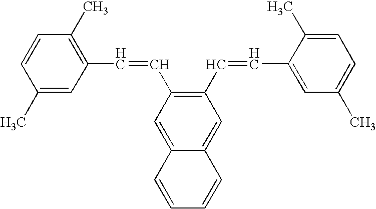 |
| 2-07 | 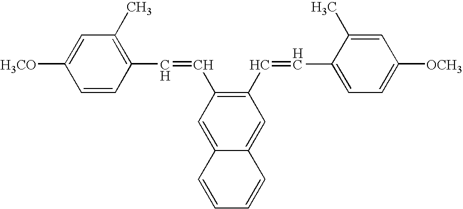 |
| 2-08 | 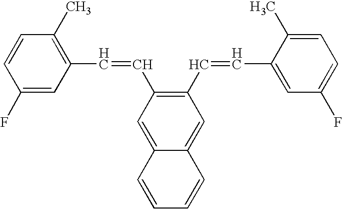 |

TABLE 38

| Compound | |
|---|---|
| No. | Formula |
| 2-09 | 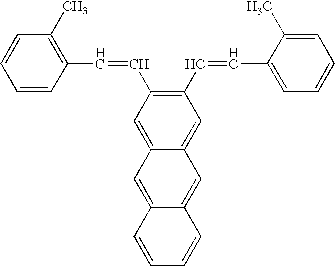 |
| 2-10 | 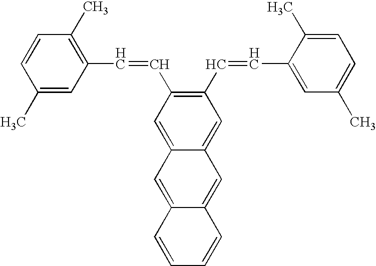 |
| 2-11 | 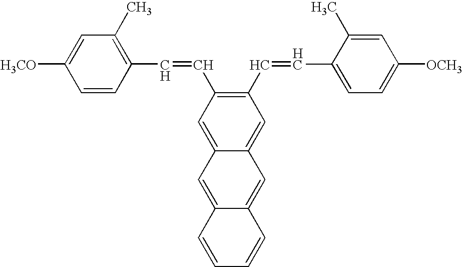 |
| 2-12 | 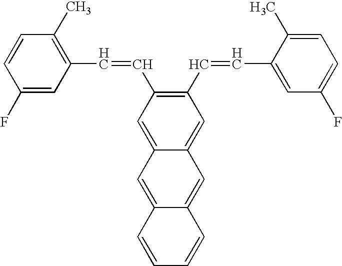 |

TABLE 39

| Compound No. | Formula |
|---|---|
| 2-13 | 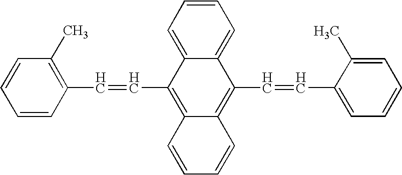 |
| 2-14 | 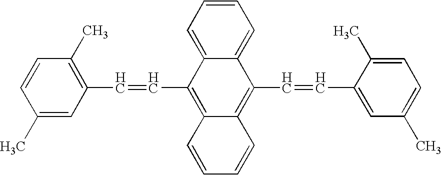 |
| 2-15 | 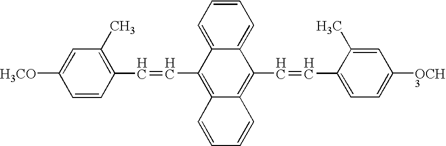 |
| 2-16 | 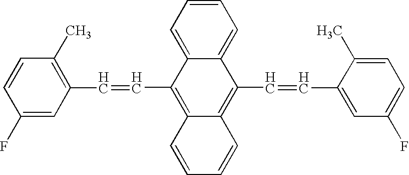 |

TABLE 40

| Compound No. | Formula |
|---|---|
| 2-17 | 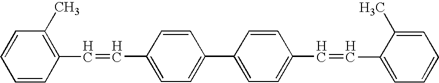 |
| 2-18 | 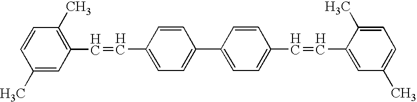 |
| 2-19 | 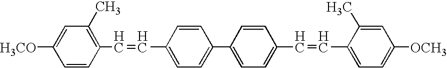 |
| 2-20 | 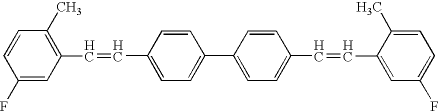 |

The organic electroluminescent element of the present invention comprises a layer containing the compound represented by the formula (1). The organic electroluminescent element of the present invention may be in a variety of embodiments, and may basically have a pair of electrodes (cathode and anode), and a luminescence layer containing the compound represented by the formula (1). Further, the element may optionally have a positive hole-transporting layer and an electron-transporting layer, which may further improve the luminescent property of the element in most of cases. The organic electroluminescent element of the present invention may preferably comprise a substrate for supporting the layers.

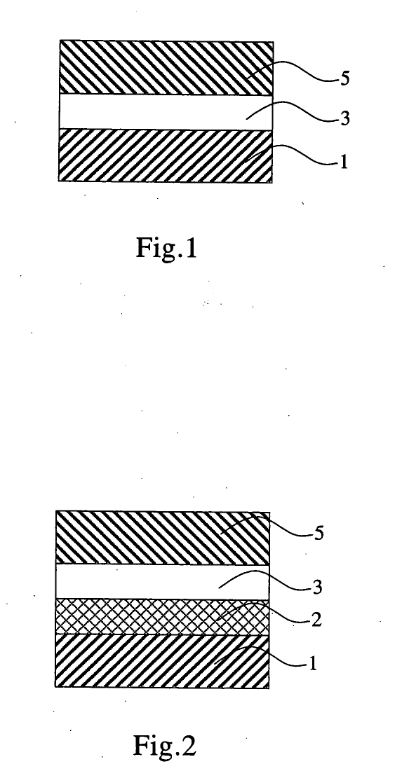

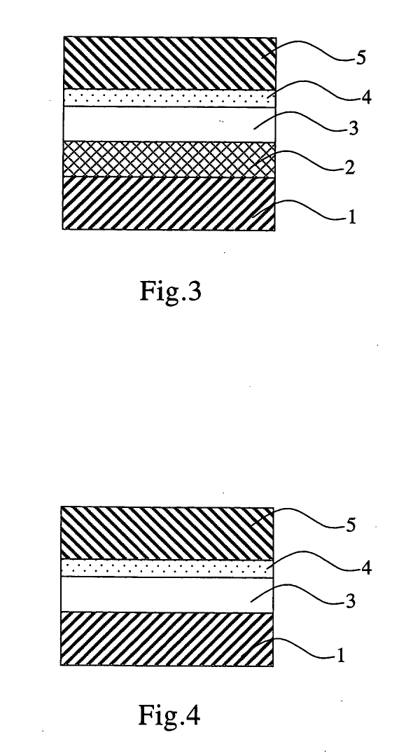

Embodiments of the organic electroluminescent element of the present invention may specifically include (1) an element of the structure shown in FIG. 1 having an anode, a luminescent layer and a cathode, (2) an element of the structure shown in FIG. 2 having an anode, a positive hole-transporting layer, a luminescent layer and a cathode, (3) an element of the structure shown in FIG. 3 having an anode, a positive hole-transporting layer, a luminescent layer, an electron-transporting layer and a cathode, and (4) an element of the structure shown in FIG. 4 having an anode, a luminescent layer, an electron-transporting layer and a cathode.

There is no particular limitation as to the substrate. For example, a glass, a transparent plastic or a silica may be used as the substrate. The material, thickness and shape of the substrate may suitably be selected or determined depending on the requirements for the construction of the element.

The anode maybe of a metal, an alloy, an electro conductive substance or combinations thereof having a relatively high work function. Examples of such electrodes may include metals such as Au, and dielectric transparent materials such as CuI, ITO, SnO₂ and ZnO. The anode may usually be produced by vapor deposition or sputtering to be in the form of a thin layer. The sheet resistivity as an electrode may preferably be several hundreds of ohms per square, or less. The thickness of the anode may depend on the material thereof and usually be selected in a range of about 10 nm to 500 nm, and preferably 20 nm to 300 nm.

The cathode may be of a metal, an alloy, an electro conductive substance or combinations thereof having a relatively low work function. Examples of such electrodes may include sodium, sodium-potassium alloy, magnesium, lithium, magnesium/copper mixture, Al/AlO₂, and indium. Similar to the anode, the cathode may also be produced by vapor deposition or sputtering to be in the form of a thin layer. The sheet resistivity as an electrode may preferably be several hundreds of ohms per square, or less. The thickness of the cathode may usually be selected in a range of about 50 nm to 1000 nm, and preferably 100 nm to 500 nm.

The positive hole-transporting layer is a layer consisting of a positive hole-transporting compound, and has a function for transporting and injecting into the luminescent layer a positive hole that has been injected from the anode. In addition to the injecting and transporting function, the positive hole-transporting layer may further have other functions such as shielding function. There is no particular limitation as to the positive hole-transporting compound as long as it has the aforementioned function. The compound may arbitrarily be selected from various organic or inorganic materials such as those previously employed as positive hole-transporting compounds in organic photoconductive materials, and those publicly known as a positive hole-transporting compounds in an organic electroluminescent element. Examples of the organic material for use as the positive hole-transporting compound may include a triazole derivative, an oxadiazole derivative, an imidazole derivative, a polyaryl alkane derivative, a pyrazoline derivative, a pyrazolone derivative, a phenylenediamine derivative, an arylamine derivative, an amino-substituted chalcone derivative, an oxazole derivative, a styrylanthracene derivative, a fluorenone derivative, a hydrazone derivative, a stilbene derivative, a ] porphyrin derivative, an aromatic tertiary amine derivative and a styrylamine compound. Examples of the inorganic material for use as the positive hole-transporting compound may include Si, SiC, and CdS. As the positive hole-transporting layer, the element of the present invention may have only one layer containing one or more species of the positive hole-transporting compounds, or may be a plurality of layers that are laminated, each containing one or more species of the positive hole-transporting compounds. The positive hole-transporting layer may be produced by any of well-known film forming processes such as vapor deposition, sputtering or spin-coating. The film thickness thereof may usually be 10 nm to 1 μm, and preferably 20 nm to 500 nm.

The electron-transporting layer is a layer consisting of an electron-transporting compound, and has a function for transporting and injecting into the luminescent layer an electron that has been injected from the cathode. In addition to the injecting and transporting function, the electron-transporting layer may further have other functions such as shielding function. There is no particular limitation as to the electron-transporting compound as long as it has the aforementioned function. The compound may be selected from the publicly known compounds. Examples of the positive hole-transporting compound may include organic materials such as a nitro-substituted fluorenone derivative, a thiopyran dioxide derivative, a diphenoquinone derivative, an anthraquinonedimethane derivative, a fluorenylidenemethane derivative, and an anthrone derivative; and inorganic materials such as Si, SiC and CdS. As the electron-transporting layer, the element of the present invention may have only one layer containing one or more species of the electron-transporting compounds, or may be a plurality of layers that are laminated, each containing one or more species of the electron-transporting compounds. The electron-transporting layer may be produced by any of film forming processes such as vapor deposition, sputtering or spin-coating. The film thickness thereof may usually be 10 nm to 1 μm, and preferably 20 nm to 500 nm.

The luminescent layer has a function of receiving the electron and positive hole injected from the electrodes or the positive hole-transporting layer and the electron-transporting layer, and emitting light by their recombination. The compound of the formula (1) is particularly suitable for the luminescent layer, and mainly used in this layer. The luminescent layer may contain as the luminescent material only the compound of the formula (1), or may contain other luminescent material such as those publicly known in addition to the compound of the formula (1). The element of the present invention may have as the luminescent layer only one layer containing one luminescent material or a mixture of two or more of the luminescent materials, or a plurality of layers, provided that at least any one of the layers contains the compound of the formula (1).

The luminescent layer may have a so-called “guest-host” construction in which a host compound is doped with a relatively small amount of a guest compound. This construction may contribute to improvement of the luminescent efficiency and driving durability. In the guest-host luminescent layer, the luminescent mainly occurs in the guest compound. In the present element, the guest-host luminescent layer may contain the compound of the formula (1) as the guest compound and/or the host compound. In the guest-host luminescent layer, the guest compound may preferably have a smaller energy gap than the host compound, and preferably has a strong fluorescence. Such a guest compound may be the compound of the formula (1), as well as various fluorescent dyes and laser dyes, preferably a coumarin derivative or a condensed ring compound. The host compound may be the compound of the formula (1) as well as an aromatic distyryl compound and a metal complex of 8-hydroxyquinoline. Ratio of the guest compound with respect to the host compound may be in the range in which concentration quenching is avoided, and may preferably be about 0.01 to 40mol per 100 mol of the host compound. Provided that at least one layer contains the compound of the formula (1), the element of the present invention may have both one or more guest-host luminescent layers and one or more luminescent layers of other construction. The sort of the luminescent materials such as host and guest compounds in each layer and the composition ratio thereof may be the same or different.

The luminescent layer may be formed by any of the generally used film forming methods such as vapor deposition or spin coating. The thickness thereof may usually be 10 nm to 500 nm, and preferable 20 nm to 200 nm.

The compound represented by the formula (1) according to the present invention is useful as a constitutional material of an organic electroluminescent element, particularly as a luminescent material thereof. The compound represented by the formula (2) is a useful intermediate compound upon producing the compound represented by the formula (1). The compounds and the production method therefor provided by the present invention make a great contribution to production of an organic electroluminescent element having high brightness and high durability.

### EXAMPLES OF THE INVENTION

The present invention will be described more in detail with reference to the Examples, but the present invention is not limited thereto.

### Example 1

Production of Compound No. 1-02 (#1)

1.07 g of tetraethyl 1,2-dimethylnaphthalene-α,α′-diyl-diphosphonate and 1.42 g of 2′-formyl-α-phenylstilbene were dissolved in 10 ml of N,N-dimethylformamide. At 5 to 10° C., 0.65 g of potassium tert-butoxide was gradually added to the reaction over 10 minutes. The reaction mixture was then stirred at room temperature for 24 hours. 10 ml of ethanol and 10 ml of water were then added. The precipitate was recovered by filtration, washed with water and then dried to obtain 1.52 g of pale yellow powders.

The pale yellow powders were then subjected to column chromatography with silica gel as a stationary phase and mixture solvent of toluene and hexane (volume ratio 1:3) as a mobile phase, to obtain a pale yellow glass substance. The substance was then re-crystallized from the mixed solvent of chloroform and ethanol, and vacuum dried at 100° C. to obtain 0.84 g of yellow glass having a strong fluorescence (yield 49%). The elementary analysis of this product resulted in 94.00% carbon (theoretical value as compound 1-02: 94.15%), and 5.75% hydrogen (theoretical value as compound 1-02: 5.85%). In infrared absorption spectrum (KBr tablet), stretching vibration due to aromatic rings was recognized at around 1590 cm⁻¹. In proton nuclear magnetic resonance spectrum (solvent CDCl₃, internal standard TMS), ring protons of aromatic rings and alkene protons were recognized at δ=6.9 to 8.1 ppm (40H). In mass spectrum, a molecular ion peak m/z=688 was recognized. From these results, it was confirmed that the compound thus obtained was compound No.1-02.

### Example 2

Production of Compound No. 1-02 (#2)

3.04 g of diethyl diphenylmethylphosphonate and 1.94 g of 1,2-bis(2-formylstyryl)naphthalene were dissolved in 30 ml of N,N-dimethylformamide. At 5 to 10° C., 1.30 g of potassium tert-butoxide was gradually added to the reaction over 10 minutes. The reaction mixture was then stirred at room temperature for 20 hours. 80 ml of ethanol and 25 ml of water were then added. The precipitate was recovered by filtration, washed with water and then dried to obtain pale yellow powders. The pale yellow powders were treated in the same way as in Example 1, to obtain compound No.1-02.

### Example 3

Production of Compound No. 1-02 (#3)

0.921 g of 1,2-diformylnaphthalene and 4.06 g of diethyl 2-(2,2′-diphenylvinyl)benzylphosphonate were dissolved in 30 ml of N,N-dimethylformamide. At 5 to 10° C., 1.30 g of potassium tert-butoxide was gradually added to the reaction over 10 minutes. The reaction mixture was then stirred at room temperature for 20 hours. 80 ml of ethanol and 25 ml of water were then added. The precipitate was recovered by filtration, washed with water and then dried to obtain pale yellow powders. The pale yellow powders were treated in the same way as in Example 1, to obtain compound No.1-02.

### Example 4

Production of Compound No. 1-02 (#4)

A mixture consisting of 7.20 g of 1,2-bis(2-methylstyryl)naphthalene, 7.12 g of N-bromosuccinimide, 0.5 g of benzoyl peroxide containing 25% water, and 80 ml of carbon tetrachloride was heated to reflux for 10 hours. After cooling, 200 ml of diethyl ether was added to the mixture and the deposited crystals were filtered off. The filtrate liquid was concentrated to obtain a bromomethyl substance. This bromomethyl substance was admixed with 13.29 g of triethyl phosphite and heated at 120° C. for 5 hours. After finishing the reaction, generated ethyl bromide and triethyl phosphite in excess were distilled off under the reduced pressure, to obtain tetraethyl 1,2-bis(2-methylstyryl)naphthalene-α,α′-diyl-diphosphonate.

1.82 g of benzophenone and 3.16 g of tetraethyl 1,2-bis(2-methylstyryl)naphthalene-α,α′-diyl-diphosphonate obtained in the above were dissolved in 30 ml of N,N-dimethylformamide. At 5 to 10° C., 1.30 g of potassium tert-butoxide was gradually added to the reaction over 10 minutes. The reaction mixture was then stirred at room temperature for 20 hours. 80 ml of ethanol and 25 ml of water were then added. The precipitate was recovered by filtration, washed with water and then dried to obtain yellow powders. The yellow powders were treated in the same way as in Example 1, to obtain compound No.1-02.

### Example 5

Production of Compound No. 2-01 (#1)

4.28 g of tetraethyl 1,2-dimethylnaphthalene-α,α′-diyl-diphosphonate and 2.64 g of 2-methylbenzaldehyde were dissolved in 40 ml of N,N-dimethylformamide. At 5 to 10° C., 2. 60 g of potassium tert-butoxide was gradually added to the reaction over 10 minutes. Subsequently, the reaction mixture was stirred at room temperature for 20 hours, and then poured into a mixture consisting of 150 ml of water and 150 ml of ethanol. The precipitate was recovered by filtration, washed with water and then dried to obtain pale brown powders. The powders were re-crystallized from ethanol, to obtain 1.79 g of pale yellow crystals (yield 50%). The elementary analysis of this product resulted in 93.00% carbon (theoretical value as compound 2-01: 93.29%), and 6.52% hydrogen (theoretical value as compound 2-01: 6.71%). In infrared absorption spectrum (KBr tablet), stretching vibration due to aromatic rings was recognized at around 1600cm⁻¹. In mass spectrum, a molecular ion peak m/z=360 was recognized. From these results, it was confirmed that the compound thus obtained was compound No.2-01.

### Example 6

Production of Compound No. 2-01 (#2)

1.84 g of 1,2-diformylnaphthalene and 4.85 g of diethyl 2-methylbenzyl phosphonate were dissolved in 60 ml of N,N-dimethylformamide. At 5 to 10° C., 2.60 g of potassium tert-butoxide was gradually added to the reaction over 10 minutes. The reaction mixture was then stirred at room temperature for 20 hours. 150 ml of water was then added. The precipitate was recovered by filtration, washed with water and then dried to obtain pale brown powders. The powders were treated in the same way as in Example 5, to obtain compound No.2-01.

### Example 7

On a glass substrate on which a thin layer of indium tin oxide that is a transparent electrode was previously formed as an anode (ITO glass substrate), a positive hole-transporting layer, a luminescent layer, an electron-transporting layer and an aluminum/lithium (Al/Li) electrode as a cathode were formed, to produce the organic electroluminescent element of the present invention. Specifically, an ITO glass substrate, N,N′-diphenyl-N,N′-bis(3-methylphenyl)benzidine (TPD) as apositive hole-transporting material, the aromatic methylidene compound No.1-02 according to the present invention as a luminescent material, and tris(8-hydroxyquinolino)aluminum as an electron transporting material were placed in a vacuum vapor deposition system. The air was drawn out to 10⁻⁴ Pa. On the electrode of the ITO glass substrate, TPD, the positive hole-transporting material, was vapor-deposited at the deposition rate of 0.1 to 0.5nm/sec., to form a positive hole-transporting layer having a thickness of 50 nm. Subsequently, compound No.1-02, the luminescent material, was vapor-deposited at the deposition rate of 0.1 to 0.5nm/sec., to form a luminescent layer having a thickness of 50 nm. Alq, the electron-transporting material, was then deposited at the rate of 0.1 nm/sec., to form an electron transporting layer having a thickness of 10 nm. Further, Li and Al were simultaneously deposited at the deposition rate of 0.01 to 0.02 nm/sec. and 1 to 2 nm/sec., respectively, to form an Al/Li electrode having a thickness of 150 nm. These vapor deposition steps were performed continuously keeping the vacuum. The thickness of each layer was monitored with a quartz oscillator for controlling the thickness. After forming all of the layers, the electrode was immediately taken out in a dry nitrogen atmosphere, to produce the organic electroluminescent element. Application of a voltage to the element thus produced resulted in uniform blue luminescent.

### Example 8

Production of Compound No. 1-22 (#1)

1.07 g of tetraethyl 2,3-dimethylnaphthalene-α,α′-diyl-diphosphonate and 1.42 g of 2′-formyl-a-phenylstilbene were dissolved in 10 ml of N,N-dimethylformamide. At 5 to 10° C., 0.65 g of potassium tert-butoxide was gradually added to the reaction over 10 minutes. The reaction mixture was then stirred at room temperature for 24 hours. 10 ml of ethanol and 10 ml of water were then added. The precipitate was recovered by filtration, washed with water and then dried to obtain pale yellow powders.

The pale yellow powders were then subjected to Column chromatography with silica gel as a stationary phase and toluene as a mobile phase, to obtain a pale yellow glass substance. The substance was then re-crystallized from the mixed solvent of toluene and hexane, and vacuum dried at 100° C. to obtain 0.80 g of pale yellow crystals having a strong fluorescence (yield 47%). The melting point thereof was 208.0 to 212.0° C. The elementary analysis of this product resulted in 94.01% carbon (theoretical value as compound 1-22: 94.15%), and 5.80% hydrogen (theoretical value as compound 1-22: 5.85%). In infrared absorption spectrum (KBr tablet), stretching vibration due to aromatic rings was recognized at around 1595cm⁻¹. In proton nuclear magnetic resonance spectrum (solvent CDCl₃, internal standard TMS), ring protons of aromatic rings and alkene protons were recognized at δ=6.9 to 7.9 ppm (40H). In mass spectrum, a molecular ion peak m/z=688 was recognized. From these results, it was confirmed that the compound thus obtained was compound No.1-22.

### Example 9

Production of Compound No. 1-22 (#2)

3.04 g of diethyl diphenylmethylphosphonate and 1.94 g of 2,3-bis(2-formylstyryl)naphthalene were dissolved in 30 ml of N,N-dimethylformamide. At 5 to 10° C., 1.30 g of potassium tert-butoxide was gradually added to the reaction over 10 minutes. The reaction mixture was then stirred at room temperature for 20 hours. 80 ml of ethanol and 25 ml of water were then added. The precipitate was recovered by filtration, washed with water and then dried to obtain pale yellow powders. The pale yellow powders were treated in the same way as in Example 8, to obtain compound No.1-22.

### Example 10

Production of Compound No. 1-22 (#3)

0.921 g of 2,3-diformylnaphthalene and 4.06 g of diethyl 2-(2,2′-diphenylvinyl)benzylphosphonate were dissolved in 30 ml of N,N-dimethylformamide. At 5 to 10° C., 1.30 g of potassium tert-butoxide was gradually added to the reaction over 10 minutes. The reaction mixture was then stirred at room temperature for 20 hours. 80 ml of ethanol and 25 ml of water were then added. The precipitate was recovered by filtration, washed with water and then dried to obtain pale yellow powders. The pale yellow powders were treated in the same way as in Example 8, to obtain compound No.1-22.

### Example 11

Production of Compound No. 1-22 (#4)

A mixture consisting of 7.20 g of 2,3-bis(2-methylstyryl)naphthalene, 7.12 g of N-bromosuccinimide, 0.5 g of benzoyl peroxide containing 25% water, and 80 ml of carbon tetrachloride was heated to reflux for 10 hours. After cooling, 200 ml of diethyl ether was added to the mixture and the deposited crystals were filtered off. The filtrate liquid was concentrated to obtain a bromomethyl substance. This bromomethyl substance was admixed with 13.29 g of triethyl phosphite and heated at 120° C. for 5 hours. After finishing the reaction, generated ethyl bromide and triethyl phosphite in excess were distilled off under the reduced pressure, to obtain tetraethyl 2,3-bis(2-methylstyryl)naphthalene-α,α′-diyl-diphosphonate.

1.82 g of benzophenone and 3.16 g of tetraethyl 2,3-bis(2-methylstyryl)naphthalene-α,α′-diyl-diphosphonate obtained in the above were dissolved in 30 ml of N,N-dimethylformamide. At 5 to 10° C., 1.30 g of potassium tert-butoxide was gradually added to the reaction over 10 minutes. The reaction mixture was then stirred at room temperature for 20 hours. 80 ml of ethanol and 25 ml of water were then added. The precipitate was recovered by filtration, washed with water and then dried to obtain pale yellow powders. The pale yellow powders were treated in the same way as in Example 8, to obtain compound No.1-22.

### Example 12

Production of Compound No. 2-05 (#1)

4.28 g of tetraethyl 2,3-dimethylnaphthalene-α,α′-diyl-diphosphonate and 2.64 g of 2-methylbenzaldehyde were dissolved in 40 ml of N,N-dimethylformamide. At 5 to 10° C., 2.60 g of potassium tert-butoxide was gradually added to the reaction over 10 minutes. Subsequently, the reaction mixture was stirred at room temperature for 20 hours, and then poured into a mixture consisting of 150 ml of water and 150 ml of ethanol. The precipitate was recovered by filtration, washed with water and then dried to obtain pale brown powders. The powders were re-crystallized from ethanol, to obtain 1.50 g of pale yellow crystals (yield 42%). The elementary analysis of this product resulted in 93.22% carbon (theoretical value as compound 2-05: 93.29%), and 6.63% hydrogen (theoretical value as compound 2-05: 6.71%). In infrared absorption spectrum (KBr tablet), stretching vibration due to aromatic rings was recognized at around 1600 cm⁻¹. In mass spectrum, a molecular ion peak m/z=360 was recognized. From these results, it was confirmed that the compound thus obtained was compound No.2-05.

### Example 13

Production of Compound No. 2-05 (#2)

1.84 g of 2,3-diformylnaphthalene and 4.85 g of diethyl 2-methylbenzyl phosphonate were dissolved in 60 ml of N,N-dimethylformamide. At 5 to 10° C., 2.60 g of potassium tert-butoxide was gradually added to the reaction over 10 minutes. The reaction mixture was then stirred at room temperature for 20 hours. 150 ml of water was then added. The precipitate was recovered by filtration, washed with water and then dried to obtain pale brown powders. The powders were treated in the same way as in Example 12, to obtain compound No.2-05.

### Example 14

An organic electroluminescent element was produced in the same way as in Example 7 except that the aromatic methylidene compound No.1-22 according to the present invention was employed as a luminescent material. Application of a voltage to the element thus produced resulted in uniform blue luminescent.

### Example 15

Production of Compound No. 1-42 (#1)

1.20 g of tetraethyl 2,3-dimethylanthracene-α,α′-diyl-diphosphonate and 1.42 g of 2′-formyl-α-phenylstilbene were dissolved in 10 ml of N,N-dimethylformamide. At 5 to 10° C., 0.65 g of potassium tert-butoxide was gradually added to the reaction over 10 minutes. The reaction mixture was then stirred at room temperature for 24 hours. 10 ml of ethanol and 10 ml of water were then added. The precipitate was recovered by filtration, washed with water and then dried to obtain 1.35 g of yellowish brown powders.

The yellowish brown powders were then subjected to column chromatography with silica gel as a stationary phase and toluene as a mobile phase, to obtain an orange-yellow glass substance. The substance was then re-crystallized from the mixed solvent of toluene and hexane, and vacuum dried at 100° C. to obtain 1.10 g of pale orange crystals (yield 59%). The elementary analysis of this product resulted in 94.10% carbon (theoretical value as compound 1-42: 94.27%), and 5.56% hydrogen (theoretical value as compound 1-42: 5.73%). In infrared absorption spectrum (KBr tablet), stretching vibration due to aromatic rings was recognized at around 1600 cm⁻¹. In proton nuclear magnetic resonance spectrum (solvent DMSO-d₆, internal standard TMS), ring protons of aromatic rings and alkene protons were recognized at δ=6.9 to 8.5 ppm (42H). In mass spectrum, a molecular ion peak m/z=738 was recognized. From these results, it was confirmed that the compound thus obtained was compound No.1-42.

### Example 16

Production of Compound No. 1-42 (#2)

3.04 g of diethyl diphenylmethylphosphonate and 2.19 g of 2,3-bis(2-formylstyryl)anthracene were dissolved in 30 ml of N,N-dimethylformamide. At 5 to 10° C., 1.30 g of potassium tert-butoxide was gradually added to the reaction over 10 minutes. The reaction mixture was then stirred at room temperature for 20 hours. 80 ml of ethanol and 25 ml of water were then added. The precipitate was recovered by filtration, washed with water and then dried to obtain yellowish brown powders. The yellowish brown powders were treated in the same way as in Example 15, to obtain compound No.1-42.

### Example 17

Production of Compound No. 1-42 (#3)

0.703 g of 2,3-diformylanthracene and 2.56 g of diethyl 2-(2,2′-diphenylvinyl)benzylphosphonate were dissolvedin 20 ml of N,N-dimethylformamide. At 5 to 10° C., 0.78 g of potassium tert-butoxide was gradually added to the reaction over 10 minutes. The reaction mixture was then stirred at room temperature for 20 hours. 80 ml of ethanol was then added. The precipitate was recovered by filtration, washed with water and then dried to obtain yellowish brown powders. The yellowish brown powders were treated in the same way as in Example 15, to obtain compound No.1-42.

### Example 18

Production of Compound No. 1-42 (#4)

A mixture consisting of 8.21 g of 2,3-bis(2-methylstyryl)anthracene, 7.12 g of N-bromosuccinimide, 0.5 g of benzoyl peroxide containing 25% water, and 80 ml of carbon tetrachloride was heated to reflux for 10 hours. After cooling, 200 ml of diethyl ether was added to the mixture and the deposited crystals were filtered off. The filtrate liquid was concentrated to obtain a bromomethyl substance. This bromomethyl substance was admixed with 13.29 g of triethyl phosphite and heated at 120° C. for 5 hours. After finishing the reaction, generated ethyl bromide and triethyl phosphite in excess were distilled off under the reduced pressure, to obtain tetraethyl 2,3-bis(2-methylstyryl)anthracene-α,α′-diyl-diphosphonate.

1.82 g of benzophenone and 3.41 g of tetraethyl 2,3-bis(2-methylstyryl)anthracene-α,α′-diyl-diphosphonate obtained in the above were dissolved in 30 ml of N,N-dimethylformamide. At 5 to 10° C., 1.30 g of potassium tert-butoxide was gradually added to the reaction over 10 minutes. The reaction mixture was then stirred at room temperature for. 20 hours. 80 ml of ethanol was then added. The precipitate was recovered by filtration, washed with water and then dried to obtain yellowish brown powders. The yellowish brown powders were treated in the same way as in Example 15, to obtain compound No.1-42.

### Example 19

Production of Compound No. 2-09 (#1)

4.78 g of tetraethyl 2,3-dimethylanthracene-α,α′-diyl-diphosphonate and 2.64 g of 2-methylbenzaldehyde were dissolved in 40 ml of N,N-dimethylformamide. At 5 to 10° C., 2.60 g of potassium tert-butoxide was gradually added to the reaction over 10 minutes. Subsequently, the reaction mixture was stirred at room temperature for 20 hours, and then poured into a mixture consisting of 150 ml of water and 150 ml of ethanol. The precipitate was recovered by filtration, washed with water and then dried to obtain brown powders. The powders were re-crystallized from toluene, to obtain 2.05 g of pale brown crystals (yield 50%). The elementary analysis of this product resulted in 93.54% carbon (theoretical value as compound 2-09: 93.62%), and 6.26% hydrogen (theoretical value as compound 2-09: 6.38%). In infrared absorption spectrum (KBr tablet)-, stretching vibration due to aromatic rings was recognized at a round 1600 cm⁻¹. In mass spectrum, a molecular ion peak m/z=410 was recognized. From these results, it was confirmed that the compound thus obtained was compound No.2-09.

### Example 20

Production of Compound No. 2-09 (#2)

2.34 g of 1,2-diformylanthracene and 4.85 g of diethyl 2-methylbenzyl phosphonate were dissolved in 60 ml of N,N-dimethylformamide. At 5 to 10° C., 2.60 g of potassium tert-butoxide was gradually added to the reaction over 10 minutes. The reaction mixture was then stirred at room temperature for 20 hours. 150 ml of water was then added. The precipitate was recovered by filtration, washed with water and then dried to obtain brown powders. The powders were treated in the same way as in Example 19, to obtain compound No.2-09.

### Example 21

An organic electroluminescent element was produced in the same way as in Example 7 except that the aromatic methylidene compound No.1-42 according to the present invention was employed as a luminescent material. Application of a voltage to the element thus. produced resulted in uniform green luminescent.

### Example 22

Production of Compound No. 1-62 (#1)

1.20 g of tetraethyl 9,10-dimethylanthracene-α,α′-diyl-diphosphonate and 1.42 g of 2′-formyl-α-phenylstilbene were dissolved in 10 ml of N,N-dimethylformamide. At 5 to 10° C., 0.65 g of potassium tert-butoxide was gradually added to the reaction over 10 minutes. The reaction mixture was then stirred at roomtemperature for 24 hours. 30ml of ethanol wasthenadded. The precipitate was recovered by filtration, washed with water and then dried to obtain 1.42 g of yellow powders.

The yellow powders were then subjected to column chromatography with silica gel as a stationary phase and toluene as a mobile phase, to obtain a purified yellow powders. The powders were then re-crystallized from toluene, and vacuum dried at 100° C. to obtain 1.25 g of yellow crystals (yield 68%). The elementary analysis of this product resulted in 94.12% carbon (theoretical value as compound 1-62: 94.27%), and 5.56% hydrogen (theoretical value as compound 1-62: 5.73%). In infrared absorption spectrum (KBr tablet), stretching vibration due to aromatic rings was recognized at around 1600 cm⁻¹. In proton nuclear magnetic resonance spectrum (solvent CDCl₃, internal standard TMS), ring protons of aromatic rings and alkene protons were recognized at δ=6.9 to 8.4 ppm (42H). In mass spectrum, a molecular ion peak m/z=738 was recognized. From these results, it was confirmed that the compound thus obtained was compound No.1-62.

### Example 23

Production of Compound No. 1-62 (#2)

3.04 g of diethyl diphenylmethylphosphonate and 2.19 g of 9,10-bis(2-formylstyryl)anthracene were dissolved in 30 ml of N,N-dimethylformamide. At 5 to 10° C., 1.30 g of potassium tert-butoxide was gradually added to the reaction over 10 minutes. The reaction mixture was then stirred at room temperature for 20 hours. 80 ml of ethanol was then added. The precipitate was recovered by filtration, washed with water and then dried to obtain yellow powders. The yellow powders were treated in the same way as in Example 22, to obtain compound No.1-62.

### Example 24

Production of Compound No. 1-62 (#3)

1.17 g of 9,10-diformylanthracene and 4.06 g of diethyl 2- (2,2′-diphenylvinyl)benzylphosphonate were dissolvedin 30 ml of N,N-dimethylformamide. At 5 to 10° C., 1.30 g of potassium tert-butoxide was gradually added to the reaction over 10 minutes. The reaction mixture was then stirred at room temperature for 20 hours. 80 ml of ethanol was then added. The precipitate was recovered by filtration, washed with water and then dried to obtain yellow powders. The yellow powders were treated in the same way as in Example 22, to obtain compound No.1-62.

### Example 25

Production of Compound No. 1-62 (#4)

A mixture consisting of 8.21 g of 9,10-bis(2-methylstyryl)anthracene, 7.12 g of N-bromosuccinimide, 0.5 g of benzoyl peroxide containing 25% water, and 80 ml of carbon tetrachloride was heated to reflux for 10 hours. After cooling, 200 ml of diethyl ether was added to the mixture and the deposited crystals were filtered off. The filtrate liquid was concentrated to obtain a bromomethyl substance. This bromomethyl substance was admixed with 13.29 g of triethyl phosphite and heated at 120° C. for 5 hours. After finishing the reaction, generated ethyl bromide and triethyl phosphite in excess were distilled off under the reduced pressure, to obtain tetraethyl 9,10-bis(2-methylstyryl)anthracene-α,α′-diyl-diphosphonate.

1.82 g of benzophenone and 3.41 g of tetraethyl 9,10-bis(2-methylstyryl)anthracene-α,α′-diyl-diphosphonate obtained in the above were dissolved in 30 ml of N,N-dimethylformamide. At 5 to 10° C., 1.30 g of potassium tert-butoxide was gradually added to the reaction over 10 minutes. The reaction mixture was then stirred at room temperature for 20 hours. Ethanol was then added. The precipitate was recovered by filtration, washed with water and then dried to obtain yellow powders. The yellow powders were treated in the same way as in Example 22, to obtain compound No.1-62.

### Example 26

Production of Compound No. 2-13 (#1)

4.78 g of tetraethyl 9,10-dimethylanthracene-α,α′-diyl-diphosphonate and 2.64 g of 2-methylbenzaldehyde were dissolved in 40 ml of N,N-dimethylformamide. At 5 to 10° C., 2.60 g of potassium tert-butoxide was gradually added to the reaction over 10 minutes. Subsequently, the reaction mixture was stirred at room temperature for 20 hours, and then poured into a mixture consisting of 150 ml of water and 150 ml of ethanol. The precipitate was recovered by filtration, washed with water and then dried to obtain brown powders. The powders were re-crystallized toluene, to obtain 2.23 g of pale brown crystals (yield 54%). The elementary analysis of this product resulted in 93.51% carbon (theoretical value as compound 2-13: 93.62%), and 6.40% hydrogen (theoretical value as compound 2-13: 6.38%). In infrared absorption spectrum (KBr tablet), stretching vibration due to aromatic rings was recognized at around l600cm¹. In mass spectrum, a molecular ion peak m/z=410 was recognized. From these results, it was confirmed that the compound thus obtained was compound No.2-13.

### Example 27

Production of Compound No. 2-13 (#2).

2.354 g of 9,10-diformylanthracene and 4.85 g of diethyl 2-methylbenzyl phosphonate were dissolved in 60 ml of N,N-dimethylformamide. At 5 to 10° C., 2.60 g of potassium tert-butoxide was gradually added to the reaction over 10 minutes. The reaction mixture was then stirred at room temperature for 20 hours. 150 ml of water was then added. The precipitate was recovered by filtration, washed with water and then dried to obtain brown powders. The powders were treated in the same way as in Example 26, to obtain compound No.2-13.

### Example 28

An organic electroluminescent element was produced in the same way as in Example 7 except that the aromatic methylidene compound No.1-62 according to the present invention was employed as a luminescent material. Application of a voltage to the element thus produced resulted in uniform green luminescent.

### Example 29

Production of Compound No. 1-82 (#1)

1.13 g of tetraethyl 4,4′-dimethylbiphenyl-α,α′-diyl-diphosphonate and 1.42 g of 2′-formyl-α-phenylstilbene were dissolved in 10 ml of N,N-dimethylformamide. At 5 to 10° C., 0.65 g of potassium tert-butoxide was gradually added to the reaction over 10 minutes. The reaction mixture was then stirred at room temperature for 24 hours. 30 ml of ethanol was then added. The precipitate was recovered by filtration, washed with water and then dried to obtain pale yellow powders.

The pale yellow powders were then re-crystallized from toluene, to obtain 0.82 g of pale yellow needle crystals (yield 79%). The melting point thereof was 242.0 to 247.0° C. The elementary analysis of this product resulted in 93.89% carbon (theoretical value as compound 1-82: 94.08%), and 5.81% hydrogen (theoretical value as compound 1-82: 5.92%). In infrared absorption spectrum (KBr tablet), stretching vibration due to aromatic rings was recognized at around 1590 cm⁻¹. In proton nuclear magnetic resonance spectrum (solvent CDCl₃, internal standard TMS), ring protons of aromatic rings and alkene protons were recognized at δ=6.9 to 7.8 ppm (42H). In mass spectrum, a molecular ion peak m/z=714 was recognized. From these results, it was confirmed that the compound thus obtained was compound No.1-82.

### Example 30

Production of Compound No. 1-82 (#2)

3.04 g of diethyl diphenylmethylphosphonate and 2.07 g of 4,4′-bis(2-formylstyryl)biphenyl were dissolved in 30 ml of N,N-dimethylformamide. At 5 to 10° C., 1.30 g of potassium tert-butoxide was gradually added to the reaction over 10 minutes. The reaction mixture was then stirred at room temperature for 20 hours. 80 ml of ethanol was then added. The precipitate was recovered by filtration, washed with water and then dried to obtain pale yellow powders. The pale yellow powders were treated in the same way as in Example 29, to obtain compound No.1-82.

### Example 31

Production of Compound No. 1-82 (#3)

1.05 g of 4,4′-diformylbiphenyl and 4.06 g of diethyl 2- (2,2′-diphenylvinyl) benzylphosphonate were dissolved in 30 ml of N,N-dimethylformamide. At 5 to 10° C., 1.30 g of potassium tert-butoxide was gradually added to the reaction over 10 minutes. The reaction mixture was then stirred at room temperature for 20 hours. 80 ml of ethanol was then added. The precipitate was recovered by filtration, washed with water and then dried to obtain pale yellow powders. The pale yellow powders were treated in the same way as in Example 29, to obtain compound No.1-82.

### Example 32

Production of Compound No. 1-82 (#4)

A mixture consisting of 7.73 g of 4,4′-bis(2-methylstyryl)biphenyl, 7.12 g of N-bromosuccinimide, 0.5 g of benzoyl peroxide containing 25% water, and 80 ml of carbon tetrachloride was heated to reflux for 10 hours. After cooling, 200 ml of diethyl ether was added to the mixture and the deposited crystals were filtered off. The filtrate liquid was concentrated to obtain a bromomethyl substance. This bromomethyl substance was admixed with 13.29 g of triethyl phosphite and heated at 120° C. for 5 hours. After finishing the reaction, generated ethyl bromide and triethyl phosphite in excess were distilled off under the reduced pressure, to obtain tetraethyl 4,4′-bis(2-methylstyryl)biphenyl-α,α′-diyl-diphosphonate.

1.82 g of benzophenone and 3.29 g of tetraethyl 4,4′-bis(2-methylstyryl)biphenyl-α,α′-diyl-diphosphonate obtained in the above were dissolved in 30 ml of N,N-dimethylformamide. At 5 to 10° C., 1.30 g of potassium tert-butoxide was gradually added to the reaction over 10 minutes. The reaction mixture was then stirred at room temperature for 20 hours. 80 ml of ethanol was then added. The precipitate was recovered by filtration, washed with water and then dried to obtain pale yellow powders. The pale yellow powders were treated in the same way as in Example 29, to obtain compound No.1-82.

### Example 33

Production of Compound No. 2-17 (#1)

4.54 g of tetraethyl 4,4′-dimethylbiphenyl-α,α′-diyl-diphosphonate and 2.64 g of 2-methylbenzaldehyde were dissolved in 40 ml of N,N-dimethylformamide. At 5 to 10° C., 2.60 g of potassium tert-butoxide was gradually added to the reaction over 10 minutes. Subsequently, the reaction mixture was stirred at room temperature for 20 hours, and then poured into a mixture consisting of 150 ml of water and 150 ml of ethanol. The precipitate was recovered by filtration, washed with water and then dried to obtain pale yellow crystals. The crystals were re-crystallized from a mixed solvent of toluene and ethanol, to obtain 1.88 g of pale yellow crystals (yield 49%). The elementary analysis of this product resulted in 93.09% carbon (theoretical value as compound2-17: 93.22%), and 6.60% hydrogen (theoretical value as compound 2-17: 6.78%). In infrared absorption spectrum (KBr tablet), stretching vibration due to aromatic rings was recognized at around 1600 cm⁻¹. In mass spectrum, a molecular ion peak m/z=386 was recognized. From these results, it was confirmed that the compound thus obtained was compound No.2-17.

### Example 34

Production of Compound No. 2-17 (#2)

2.10 g of 4,4′-diformylbiphenyl and 4.85 g of diethyl 2-methylbenzyl phosphonate were dissolved in 60 ml of N,N-dimethylformamide. At 5 to 10° C., 2.60 g of potassium tert-butoxide was gradually added to the reaction over 10 minutes. The reaction mixture was then stirred at room temperature for 20 hours. 150 ml of water was then added. The precipitate was recovered by filtration, washed with water and then dried to obtain pale yellow crystals. The crystals were treated in the same way as in Example 33, to obtain compound No.2-17.

### Example 35

An organic electroluminescent element was produced in the same way as in Example 7 except that the aromatic methylidene compound No.1-82 according to the present invention was employed as a luminescent material. Application of a voltage to the element thus produced resulted in uniform blue luminescent.

### Example 36

Production of Compound No. 1-102 (#1)

0.671 g of phthalaldehyde and 4.06 g of diethyl 2-(2,2′-diphenylvinyl)benzylphosphonate were dissolved in 20 ml of N,N-dimethylformamide. At 5 to 10° C., 1.30 g of potassium tert-butoxide was gradually added to the reaction over 10 minutes. The reaction mixture was then stirred at room temperature for 60 hours. Subsequently, 5 ml of acetic acid was added, and the reaction mixture was then poured into 200 ml of water. The precipitate was recovered by filtration, washed with water, washed with ethanol, and then dried to obtain white powders having slight brown color. The powders were then subjected to column chromatography with silica gel as a stationary phase and mixture solvent of toluene and hexane (volume ratio 1:1) as a mobile phase, to obtain a glass substance having slight yellow color. The substance was then re-crystallized from toluene and hexane, to obtain 1.43 g of white crystals (yield 45%). The melting point thereof was 164.5 to 165.5° C. The elementary analysis of this product resulted in 93.80% carbon (theoretical value as compound 1-102: 94.00%), and 5.82% hydrogen (theoretical value as compound 1-102: 6.00%). In infrared absorption spectrum (KBr tablet), stretching vibration due to aromatic rings was recognized at around 1600 cm⁻¹. In proton nuclear magnetic resonance spectrum (solvent CDCl₃, internal standard TMS), ring protons of aromatic rings and alkene protons were recognized at δ=6.9 to 7.6 ppm (38H). In mass spectrum, a molecular ion peak m/z=638 was recognized. From these results, it was confirmed that the compound thus obtained was compound No.1-102.

### Example 37

Production of Compound No. 1-102 (#2)

0.946 g of tetraethyl 1,2-dimethylbenzene-α,α′-diyl-diphosphonate, and 1.42 g of 2′-formyl-α-phenylstilbene were dissolved in 10 ml of N,N-dimethylformamide. At 5 to 10° C., 0.65 g g of potassium tert-butoxide was gradually added to the reaction over 10 minutes. Subsequently, the reaction mixture was stirred at room temperature for 24 hours, and then poured into 200 ml of water. The precipitate was recovered by filtration, washed with water, washed with ethanol and then dried to obtain white powders having slight brown color. The powders were treated in the same way as in Example 36, to obtain compound No.1-102.

### Example 38

Production of Compound No. 1-102 (#3)

1.82 g of benzophenone, 2.91 g of tetraethyl 1,2-bis(2-methylstyryl)benzene-α,α′-diyl-diphosphonate were dissolved in 30 ml of N,N-dimethylformamide. At 5 to 10° C., 1.30 g of potassium tert-butoxide was gradually added to the reaction over 10 minutes. Subsequently, the reaction mixture was stirred at room temperature for 20 hours, and then poured into 300 ml of water. The precipitate was recovered by filtration, washed with water, washed with ethanol and then dried to obtain white powders having slight brown color. The powders were treated in the same way as in Example 36, to obtain compound No.1-102.

### Example 39

An organic electroluminescent element was produced in the same way as in Example 7 except that the aromatic methylidene compound No.1-102 according to the present invention was employed as a luminescent material. Application of a voltage to the element thus produced resulted in uniform blue luminescent.

### Example 40

Production of Compound No. 1-122 (#1)

0.537 g of terephthalaldehyde and 3.25 g of diethyl 2- (2,2′-diphenylvinyl)benzylphosphonate were dissolved in 16 ml of N,N-dimethylformamide. At 5 to 10° C., 1.04 g of potassium tert-butoxide was gradually added to the reaction over 10 minutes. The reaction mixture was then stirred at room temperature for 60 hours. Subsequently, 30 ml of ethanol was added to the mixture. The precipitate was recovered by filtration, washed with water, washed with ethanol, and then dried to obtain pale yellow crystals. The pale yellow crystals were then re-crystallized from toluene, to obtain 1.56 g of yellow crystals (yield 61%). Melting point of these crystals was 247.0 to 252.0° C. The elementary analysis of this product resulted in 93.78% carbon (theoretical value as compound 1-122: 94.00%), and 5.91% hydrogen (theoretical value as compound 1-122: 6.00%). In infrared absorption spectrum (KBr tablet), stretching vibration due to aromatic rings was recognized at around 1590cm⁻¹. In proton nuclear magnetic resonance spectrum (solvent CDCl₃, internal standard TMS), ring protons of aromatic rings and alkene protons were recognized at δ=6.9 to 7.6 ppm (38H). In mass spectrum, a molecular ion peak m/z=638 was recognized. From these results, it was confirmed that the compound thus obtained was compound No.1-122.

### Example 41

Production of Compound No. 1-122 (#2)

0.946 g of tetraethyl 1,4-dimethylbenzene-α,α′-diyl-diphosphonate, and 1.42 g of 2′-formyl-d-phenylstilbene were dissolved in 10 ml of N,N-dimethylformamide. At 5 to 10° C., 0.65 g g of potassium tert-butoxide was gradually added to the reaction over 10 minutes. The reaction mixture was then stirred at room temperature for 24 hours. 20 ml of ethanol was then added. The precipitate was recovered by filtration, washed with water, washed with ethanol and then dried to obtain pale yellow crystals. The crystals were treated in the same way as in Example 40, to obtain compound No.1-122.

### Example 42

Production of Compound.No. 1-122 (#3)

1.82 g of benzophenone, and 2.91 g of tetraethyl 1,4-bis(2-methylstyryl)benzene-α,α′-diyl-diphosphonate were dissolved in 30 ml of N,N-dimethylformamide. At 5 to 10° C., 1.30 g of potassium tert-butoxide was gradually added to the reaction over 10-minutes. Subsequently, the reaction mixture was then stirred at room temperature for 20 hours, and then added to 40 ml of ethanol. The precipitate was recovered by filtration, washed with water, washed with ethanol and then dried to obtain pale yellow crystals. The pale yellow crystals were treated in the same way as in Example 40, to obtain compound No.1-122.

### Example 43

An organic electroluminescent element was produced in the same way as in Example 7 except that the aromatic methylidene compound No.1-122 according to the present invention was employed as a luminescent material. Application of a voltage to the element thus produced resulted in uniform light blue luminescent.

As described above, the organic electroluminescent element of the present invention having the aromatic methylidene compound of the present invention has superior luminescent property. Further, the element is stable and has long life. Therefore, the element, compound of the present invention and production method thereof is very useful in the industry.

Although the present invention has been described with reference to the preferred examples, it should be understood that various modifications and variations can be easily made by those skilled in the art without departing from the spirit of the invention. Accordingly, the foregoing disclosure should not be interpreted in a limiting sense. The present invention is limited only by the scope of the following claims.

1. (canceled)

2. (canceled)

3. A method for producing a compound represented by the following formula (1): 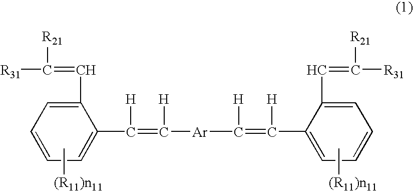
wherein
R₁₁ represents a non-substituted or substituted alkyl group, a non-substituted or substituted alkoxy group, a halogen atom, a cyano group, or a nitro group, provided that, when two or more of groups R₁₁ are present, the groups R₁₁ are the same as or different from each other,
n₁₁ is an integer of 0 to 4;
R₂₁ and R₃₁ are the same as or different from each other and each represents a hydrogen atom provided that one of R₂₁ and R₃₁ is not hydrogen atom, a non-substituted or substituted alkyl group provided that one of R₂₁ and R₃₁ is not the alkyl group, a non-substituted or substituted cycloalkyl group provided that one of R₂₁ and R₃₁ is not the cycloalkyl group, a non-substituted or substituted aromatic group, or a non-substituted or substituted heteroaromatic group; or R₂₁ and R₃₁ together form a condensed ring consisting of non-substituted or substituted aromatic rings or non-substituted or substituted heteroaromatic rings: and
Ar is selected from the groups consisting of groups represented by the following formulae: 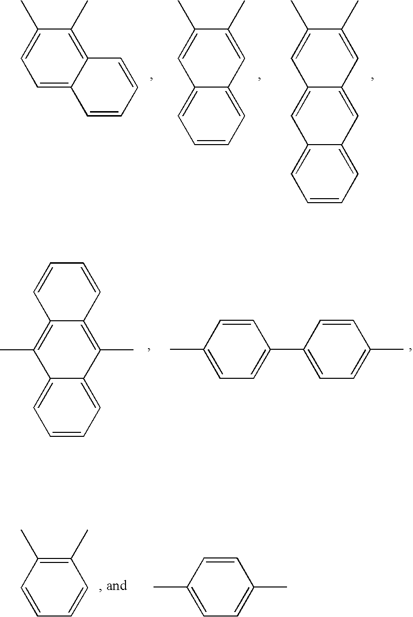
comprising the step of reacting a compound represented by the following formula (3):
ZH₂C—Ar—CH₂Z (3)
wherein Ar is the same as that in the formula (1), and groups Z are the same as or different from each other and each represents —PO(OR)₂ or —PA₃⁺ or a salt thereof, wherein R represents a non-substituted or substituted alkyl group, and A represents a non-substituted or substituted aryl group;
with a compound represented by the following formula (4) 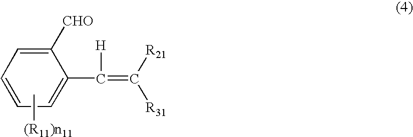
wherein R₁₁, R₂₁, R₃₁ and n₁₁ are the same as those in the formula (1).

4. A method for producing a compound represented by the following formula (1): 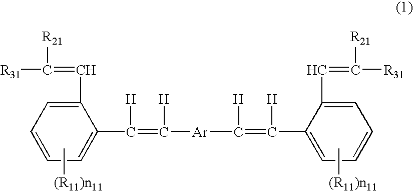
wherein
R₁₁ represents a non-substituted or substituted alkyl group, a non-substituted or substituted alkoxy group, a halogen atom, a cyano group, or a nitro group, provided that, when two or more of groups R₁₁ are present, the groups R₁₁ are the same as or different from each other:
n₁₁ is an integer of 0 to 4:
R₂₁ and R₃₁ are the same as or different from each other and each represents a hydrogen atom provided that one of R₂₁ and R₃₁ is not hydrogen atom, a non-substituted or substituted alkyl group provided that one of R₂₁ R₃₁ is not the alkyl group, a non-substituted or substituted cycloalkyl group provided that one of R₂₁ and R₃₁ is not the cycloalkyl group, a non-substituted or substituted aromatic group, or a non-substituted or substituted heteroaromatic group: or R₂₁ and R₃₁ together form a condensed ring consisting of non-substituted or substituted aromatic rings or non-substituted or substituted heteroaromatic rings: and
Ar is selected from the groups consisting of groups represented by the following formulae: 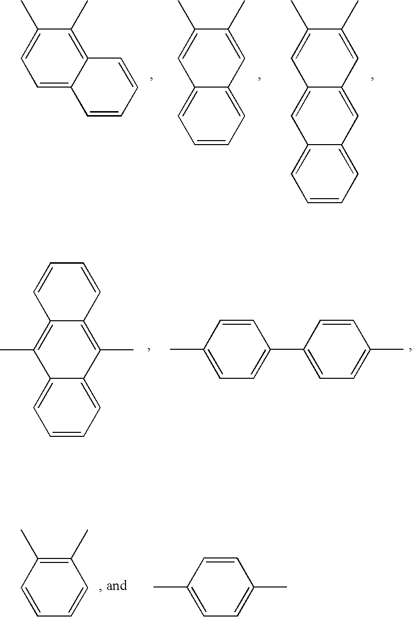
comprising the step of reacting a compound represented by the following formula (5): 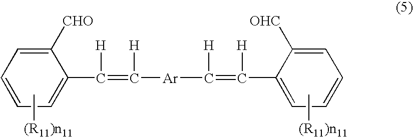
wherein R₁₁, n₁₁ and Ar are the same as those in the formula (1);
with a compound represented by the following formula (6): 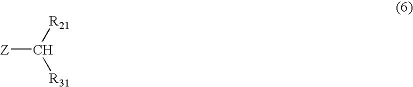
wherein R₂₁ and R₃₁ are the same as those in the formula (1), and Z represents —PO(OR)₂ or —PA₃⁺ or a salt thereof, wherein R represents a non-substituted or substituted alkyl group, and A represents a non-substituted or substituted aryl group.

5. A method for producing a compound represented by the following formula (1): 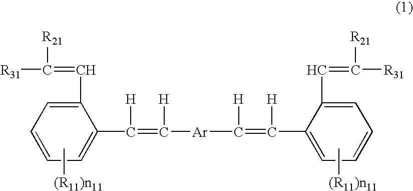
wherein
R₁₁ represents a non-substituted or substituted alkyl group, a non-substituted or substituted alkoxy group, a halogen atom, a cyano group, or a nitro group, provided that, when two or more of groups R₁₁ are present, the groups R₁₁ are the same as or different from each other:
n₁₁ is an integer of 0 to 4:
R₂₁ and R₃₁ are the same as or different from each other and each represents a hydrogen atom provided that one of R₂₁ and R₃₁ is not hydrogen atom, a non-substituted or substituted alkyl group provided that one of R₂₁ and R₃₁ is not the alkyl group, a non-substituted or substituted cycloalkyl group provided that one of R₂₁ and R₃₁ is not the cycloalkyl group, a non-substituted or substituted aromatic group, or a non-substituted or substituted heteroaromatic group: or R₂₁ and R₃₁ together form a condensed ring consisting of non-substituted or substituted aromatic rings or non-substituted or substituted heteroaromatic rings; and
Ar is selected from the groups consisting of groups represented by the following formulae: 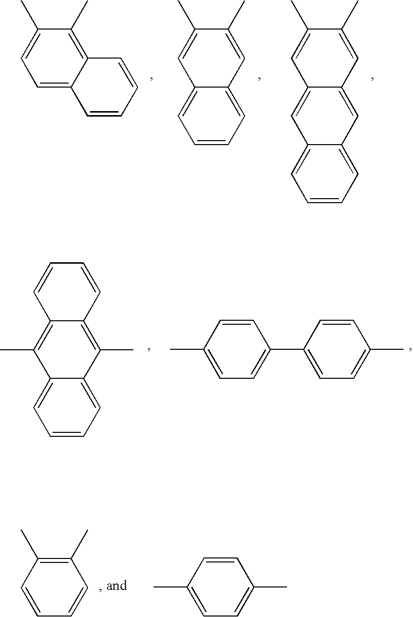
comprising the step of reacting a compound represented by the following formula (7):
OHC—Ar—CHO (7)
wherein Ar is the same as that in the formula (1); with a compound represented by the following formula (8): 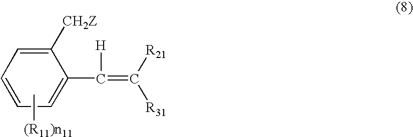
wherein R₁₁, R₂₁, R₃₁ and n₁₁ are the same as those in the formula (1), and Z represents —PO(OR)₂ or —PA₃⁺ or a salt thereof, wherein R represents a non-substituted or substituted alkyl group, and A represents a non-substituted or substituted aryl group.

6. A method for producing He a compound represented by the following formula (1) 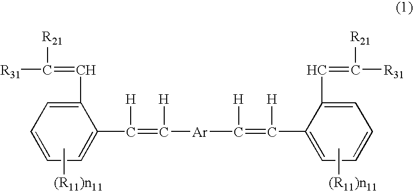
wherein
R₁₁ represents a non-substituted or substituted alkyl group, a non-substituted or substituted alkoxy group, a halogen atom, a cyano group, or a nitro group, provided that, when two or more of groups R₁₁ are present, the groups R₁₁ are the same as or different from each other:
n₁₁ is an integer of 0 to 4;
R₂₁ and R₃₁ are the same as or different from each other and each represents a hydrogen atom provided that one of R₂₁ and R₃₁ is not hydrogen atom, a non-substituted or substituted alkyl group provided that one of R₂₁ and R₃₁ is not the alkyl group, a non-substituted or substituted cycloalkyl group provided that one of R₂₁ and R₃₁ is not the cycloalkyl group, a non-substituted or substituted aromatic group, or a non-substituted or substituted heteroaromatic group; or R₂₁ and R₃₁ together form a condensed ring consisting of non-substituted or substituted aromatic rings or non-substituted or substituted heteroaromatic rings; and
Ar is selected from the groups consisting of groups represented by the following formulae: 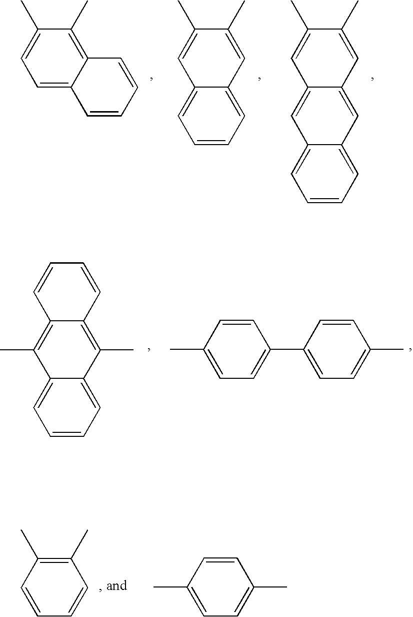
comprising the step of reacting a compound represented by the following formula (9): 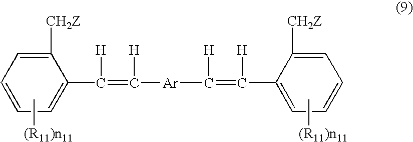
wherein R₁₁, n₁₁ and Ar is are the same as those in the formula (1), and groups Z are the same as or different from each other and each represents —PO(OR)₂ or —PA₃⁺ or a salt thereof, wherein R presents a non-substituted or substituted alkyl group, and A represents a non-substituted or substituted aryl group;
with a compound represented by the following formula (10): 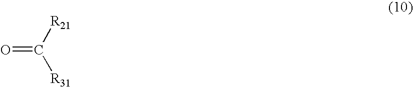
wherein R₂₁ and R₃₁ are the same as those in the formula (1).

7. A method for producing a compound represented by the following formula (2): 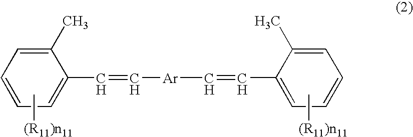
wherein R₁₁, n₁₁ and Ar are the same as those in the formula (1)
comprising the step of reacting a compound represented by the following formula (3);
ZH₂C−Ar−CH₂Z (3)
wherein Ar is the same as that in the formula (1), and groups Z are the same as or different from each other and each represents —PO(OR)₂ or —PA₃⁺ or a salt thereof, wherein R represents a non-substituted or substituted alkyl group, and A represents a non-substituted or substituted aryl group;
with a compound represented by the following formula (11): 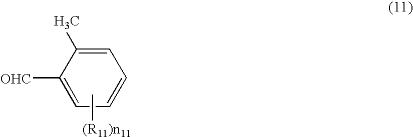
wherein R₁₁ and n₁₁ are the same as those in the formula (1).

8. A method for producing a compound represented by the following formula (2): 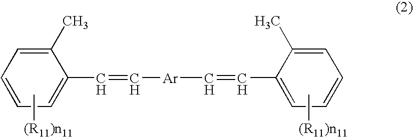
wherein R₁₁, n₁₁ and Ar are the same as those in the formula (1) comprising the steps of reacting a compound represented by the following formula (7):
OHC—Ar—CHO (7)
wherein Ar is the same as that in the formula (1); with a compound represented by the following formula (12): 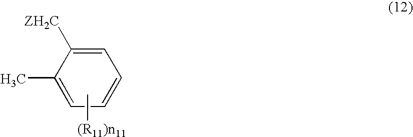
wherein R₁₁ and n₁₁ are the same as those in the formula (1); and Z represents —PO(OR)₂ or —PA₃⁺ or a salt thereof, wherein R represents a non-substituted or substituted alkyl group, and A represents a non-substituted or substituted aryl group.

9. (canceled)
